(19) United States
(12) Patent Application Publication
(10) Pub. No.: US 2025/0161495 A1
(43) Pub. Date: May 22, 2025

(54) METHODS OF GENERATING A POPULATION OF NEURONS FROM HUMAN GLIAL PROGENITOR CELLS AND GENETIC CONSTRUCTS FOR CARRYING OUT SUCH METHODS

(71) Applicant: University of Rochester, Rochester, NY (US)

(72) Inventors: Steven A. Goldman, Webster, NY (US); Abdellatif Benraiss, Pittsford, NY (US)

(73) Assignee: University of Rochester, Rochester, NY (US)

(21) Appl. No.: 18/834,925

(22) PCT Filed: Feb. 1, 2023
(86) PCT No.: PCT/US2023/061756

§ 371 Date: Jul. 31, 2024

Related U.S. Application Data
(60) Provisional application No. 63/305,570, filed on Feb. 1, 2022.

(51) Int. Cl. A61K 48/00; A61K 35/30; A61K 38/17; C12N 5/0793; C12N 9/22; C12N 15/11; C12N 15/113; C12N 15/86

(52) U.S. Cl. CPC A61K 48/0058; A61K 35/30; A61K 38/1709; A61K 38/177; C12N 5/0619; C12N 9/22; C12N 15/111; C12N 15/113; C12N 15/86; C12N 2310/141; C12N 2310/20; C12N 2310/531; C12N 2740/15043

(57) ABSTRACT

The present disclosure is directed to a method of generating a population of neurons. This method involves providing a population of human glial progenitor cells: providing a recombinant genetic construct comprising (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where the nucleic acid sequence is operably linked to the 3′ end of said promoter and/or enhancer to achieve expression of the one or more neuronal reprogramming factors; transfecting cells of the glial progenitor cell population with the recombinant genetic construct; and culturing the population after said transfecting under conditions suitable for neuron production from the transfected glial progenitor cells of the population. Also disclosed are methods of inducing the production of neurons, as well as recombinant genetic constructs.

This application claims the benefit of U.S. Provisional Patent Application Ser. No. 63/305,570, filed Feb. 1, 2022, which is hereby incorporated by reference in its entirety.

The Sequence Listing is being submitted electronically in XML format and is hereby incorporated by reference in its entirety. Said XML copy, created on Jan. 31, 2023, is named 147400-003891-SequenceListing.xml and is 24,675 bytes in size. No new matter is being introduced.

### FIELD

The present disclosure relates to methods of generating a population of neurons from human glial progenitor cells and genetic constructs for carrying out such methods.

### BACKGROUND

Neurodegenerative disorders comprise a heterogeneous category, that include both multicentric and diffuse disorders such as Alzheimer's, and those in which the loss of a single phenotype predominates, such as Huntington's and Parkinson's diseases (Goldman, S. A., “Stem and Progenitor Cell-Based Therapy of the Central Nervous System: Hopes, Hype and Wishful Thinking,” Cell Stem Cell 18(2): 174-188 (2016)). The latter category, those neuronal disorders in which a single region or phenotype is differentially affected, have proven to be the most amenable to cell type-specific neuronal replacement in animal models (Lindvall, O., “Dopaminergic Neurons for Parkinson's Therapy,” Nature Biotechnology,” 30:56-58 (2012) and Lindvall & Bjorklund, “Cell Therapeutics in Parkinson's Disease,” Neurotherapeutics: The Journal of the American Society for Experimental Neuro Therapeutics 8:539-548 (2011). These include classical Parkinson's disease, in which nigrostriatal neurons are lost before other neurons, and Huntington's disease, in which striatal atrophy becomes apparent long before the onset of more widespread cortical neuronal loss. Clinical trials of cell transplantation have already been performed for each of these prototypic neurodegenerative conditions (reviewed in (Barker et al., “Cell-Based Therapies for Parkinson Disease-Past Insights and Future Potential,” Nat. Rev. Neurol. 11:492-503 (2015); Barker et al., “The Long-Term Safety and Efficacy of Bilateral Transplantation of Human Fetal Striatal Tissue in Patients with Mild to Moderate Huntington's disease,” J. Neurol. Neurosurg. Psychiatry 84:657-665 (2013); Benraiss & Goldman, “Cellular Therapy and Induced Neuronal Replacement for Huntington's Disease,” Neurotherapeutics: The Journal of the American Society for Experimental Neuro Therapeutics 8:577-590 (2011); and Lindvall & Bjorklund, “Cell Therapeutics in Parkinson's Disease,” Neurotherapeutics: The Journal of the American Society for Experimental Neuro Therapeutics 8:539-548 (2011))). But these trials used fetal tissues dissected from the regions of interest, which thus included all cell types in the tissue, and not just the specific populations of nigrostriatal and striatal medium spiny neurons respectively lost in Parkinson's disease and Huntington's disease; in each of these cases, the target cell types typically comprised but a fraction of the cells delivered. Perhaps as a result, fetal tissue grafts into Parkinson's patients have yielded variable results, with both clear successes and failures, and a disturbingly high incidence of refractory dyskinesias, in which uncontrollable movements can negate the functional gains otherwise afforded by the grafted cells ((Barker et al., “Cell-Based Therapies for Parkinson Disease-Past Insights and Future Potential,” Nat. Rev. Neurol. 11:492-503 (2015)). Similarly, fetal striatal grafts into patients with Huntington Disease have yielded mixed results, with little evidence of significant or durable functional improvement (Cicchetti et al., “Neural Transplants in Patients with Huntington's Disease Undergo Disease-Like Neuronal Degeneration,” Proceedings of the National Academy of Sciences of the United States of America 106:12483-12488 (2009)).

Direct cellular reprogramming has allowed scientists to rapidly acquire cell types of interest for regenerative therapies and disease modeling (Lu & Yoo, “Mechanistic Insights Into MicroRNA-Induced Neuronal Reprogramming of Human Adult Fibroblasts,” Front. Neurosci. 12:522 (2018)). Several studies have demonstrated the direct conversion of human fibroblast cells to neuronal cells (see, e.g., Victor et al., “Generation of Human Striatal Neurons by microRNA-Dependent Direct Conversion of Fibroblasts,” Neuron 84(2): 311-323 (2014); U.S. Patent Application Publication No. 2002/0377885 to Yoo et al.; and Lu & Yoo, “Mechanistic Insights Into MicroRNA-Induced Neuronal Reprogramming of Human Adult Fibroblasts,” Front. Neurosci. 12:522 (2018)). Empirically, however, obtaining mature human neurons from non-neuronal cells with transcription factors has been challenging (Caiazzo et al., “Direct Generation of Functional Dopaminergic Neurons from Mouse and Human Fibroblasts,” Nature 476:224-227 (2011)).

Thus, there remains a need for the compositions and methods of producing populations of neuronal cells from populations of non-neuronal cells (e.g., glial progenitor cells).

The present application is directed to overcoming these and other deficiencies in the art.

### SUMMARY

A first aspect of the present disclosure relates to a method of generating a population of neurons. This method involves providing a population of human glial progenitor cells; providing a recombinant genetic construct comprising (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where the nucleic acid sequence is operably linked to the 3′ end of said promoter and/or enhancer to achieve expression of the one or more neuronal reprogramming factors; transfecting cells of the glial progenitor cell population with the recombinant genetic construct; and culturing the population after said transfecting under conditions suitable for neuron production from the transfected glial progenitor cells of the population.

Another aspect of the present disclosure relates to a method of inducing the production of neurons in a subject in need thereof. This method involves providing a recombinant genetic construct comprising: (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where the nucleic acid sequence is operably linked to the 3′ end of the promoter and/or enhancer. This method further involves administering, to the subject in need of neuron production, the recombinant genetic construct, under conditions effective for the one or more neuronal reprogramming factors to be expressed in glial progenitor cells of the subject, thereby inducing neuron generation in said subject.

Another aspect of the present disclosure relates to a recombinant genetic construct comprising: (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where said nucleic acid sequence is operably linked to the 3′ end of said promoter and/or enhancer to achieve expression of the one or more neuronal programming factors in glial progenitor cells.

Aspects of the present disclosure also relate to a population of human glial progenitor cells comprising a genetic construct according to the present disclosure.

Another aspect of the present disclosure relates to a method of generating a population of medium spiny neurons. This method involves providing a population of human glial progenitor cells; and expressing one or more medium spiny neuron reprogramming factors in the provided glial progenitor cell population under conditions suitable for medium spiny neuron production from the glial progenitor cells of the population.

Another aspect of the present disclosure relates to a method of generating a population of cortical interneurons. This method involves providing a population of human glial progenitor cells; and expressing one or more cortical interneuron reprogramming factors in the provided population of glial progenitor cells under conditions suitable for cortical interneuron production from the glial progenitor cells.

A number of strategies for the production of new neurons in vivo, via the induced transdifferentiation of parenchymal astrocytes, have been described. These approaches, however, are limited by the relatively limited mitotic competence of human astrocytes, such that approaches that seem efficacious in mouse models may not be expected to be feasible or effective in the human brain. In past work, it has been found that human glial progenitor cells retain neurogenic potential under appropriate conditions, and are both mitotically active and capable of undifferentiated self-renewal.

Applicant has found that human glial progenitor cells are an appropriate and effective cellular substrate for induced neuronal phenoconversion, and have designed viral vectors that achieve the efficient, cell-specific targeting of these cells, permitting the specific transduction of human glial progenitor cells with RNA binding proteins and/or transcription factors that mediate reprogramming to neuronal phenotype.

### BRIEF DESCRIPTION OF THE DRAWINGS

FIGS. 1A-1B demonstrate targeted phenoconversion of glial progenitor cells (GPCs) into neurons in adult mouse brain. FIG. 1A is a schematic of lentiviral vectors expressing shRNA against mouse Ptbp1 gene. Vectors also express EGFP in a Cre-dependent manner under the neuron-specific synapsin promoter. shRNA include shRNA 6 (CCA AAG CCT CTT TAT TCT CTT (SEQ ID NO: 3)); shRNA 8 (CAC TAT GGT TAA CTA CTA TAC (SEQ ID NO: 4)); and shRNA 9 (GGG TGA AGA TCC TGT TCA ATA (SEQ ID NO: 5)). FIG. 1B are fluorescence microscopy images from 3 weeks after treatment of 16 week-old PDGFRa-CreER mice with a mixture of the three lentiviruses from FIG. 1A. New neurons expressed the mature neuronal marker NeuN together with the PDGFRA-CreER-dependent EGFP, indicating that tehbn NeuN+ neurons arose from PDGFRA-defined glial progenitor cells.

FIGS. 2A-2B demonstrate that glial progenitor cells may be converted into medium spiny neurons by ASCL1 and BRN2 overexpression with miR 124/9. A lentiviral mixture consisting of LV-BRN2, LV-ASCL1, LV-M124/9 and LV-hSyn-DIO-EGFP was injected into the striatum of 16 week old PDGFRa-CreER mice. Mice were then treated with Tamoxifen for two weeks, to activate Cre translocation to the nucleus and recombinase-triggered GFP expression; they were sacrificed a week later, three weeks post viral injection. Histological processing the brain revealed new neurons expressing the mature neuronal marker NeuN together with EGFP, indicating the glial progenitor cell origin of these neurons, since in this system, Cre-dependent expression of EGFP can only occur in glial progenitor cells (FIG. 2A). A cohort of these newly generated, glial-derived neurons expressed DARPP2, indicative of their acquisition of a medium spiny neuronal phenotype (FIG. 2B).

FIGS. 3A-3D demonstrate that glial progenitor cells may be converted into parvalbumin interneurons, by combining direct generic differentiation of GPCs to neuronal phenotype with the addition of LMX1A and/or LHX6, transcription factors involved in the differentiation of parvalbuminergic interneurons in development. Using forced expression of Ascl, BRN2 and MIR124/9, together with either LMX1A (FIGS. 3A-3B) or LHX6 (FIGS. 3C-3D), along with fate mapping using PDGFRA-CreER-dependent neuron-specific reporter expression, we found that new parvalbumin-expressing neurons could be generated from glial progenitor cells in the mouse brain. A lentiviral mixture consisting of LV-BRN2, LV-ASCL1, LV-M124/9 and LV-hSyn-DIO-EGFP, along with either LMX1A (FIGS. 3A-3B) or LHX6 (FIGS. 3C-3D) was injected into the striatum of 16 week old PDGFRa-CreER mice. These mice were then treated with Tamoxifen for two weeks, to activate Cre translocation to the nucleus and recombinase-triggered GFP expression; they were sacrificed a week later, three weeks post viral injection. Upon processing the brains of virally-treated PDGFRa-CreER mice, new NeuN-defined neurons were readily identified by of their expression of EGFP (FIG. 3A, 3C). A cohort of these glial derived neurons also expressed Parvalbumin (PV) (FIG. 3B, FIG. 3D). Scale bar: 25 um.

FIG. 4 provides a schematic of LV-CBH-ACSL1 (left panel) and the sequence encoding human ACSL1 (SEQ ID NO: 6) (right panel).

FIG. 5 provides a schematic of LV-CBH-huBRN2 and the sequence encoding human BRN2 (SEQ ID NO: 7).

FIG. 6 provides a schematic of LV-CBH-MIR124/9 (left panel) and the sequence encoding human MIR 124/9 (SEQ ID NO: 8) (right panel).

FIG. 7 provides a schematic of LV-CBH-LMX1A and the sequence encoding human LMX1A (SEQ ID NO: 9).

FIG. 8 provides a schematic of LV-CBH-LHX6 and the sequence encoding human LHX6 (SEQ ID NO: 10).

FIG. 9 shows a schematic showing the generation of G19-PDGFRa-Cre (top panel) and the design of a lentivirus for Cre- and differentiation-dependent expression of a reporter (bottom panel).

FIG. 10 demonstrates that EGFP expression is restricted to neurons converted from human glial progenitor cells (GPCs). Briefly, G19-PDGFRa-Cre glial progenitor cells (GPCs) transduced with LV-PTBP1-shRNA-Syn-DIO-EGFP (MOI=1) (top panel). Cells were switched to neuronal media (M21-BDNF-0.5% FBS, fixed 10 days later, and imaged. Bottom panel shows synapsin-promoter restricted EGFP expression, restricted to neurons arising form human glial progenitor cells after PTBP1 knockdown mediated-phenoconversion.

FIG. 11 demonstrates synapsin promoter-restricted expression of EGFP in HuD⁺/NeuN⁺ neurons produced from PDGFRA-Cre expressing human GPCs. Cells were transduced with LV-PTBP1-shRNA 7-Syn-DIO-EGFP.

FIG. 12 demonstrates that phenoconverted neurons express mature neuronal marker MAP2. Cells were transduced with LV-PTBP1-shRNA 7-Syn-DIO-EGFP (left panel) or LV-PTBP1-shRNA 9-Syn-DIO-EGFP (right panel).

FIG. 13 demonstrates that phenoconverted neurons express mature neuronal marker Hud. Cells were transduced with LV-PTBP1-shRNA 7-Syn-DIO-EGFP (left panel) or LV-PTBP1-shRNA 9-Syn-DIO-EGFP (right panel).

FIG. 14 are graphs showing that PTBP1 knockdown selectively converts human GPCs into neurons. Left panel shows % HuC/D⁺ neurons; right panel shows % HuC/D-neurons/viral transduced cells. *Non-specific expression; the human GPC is a highly plastic cell, such that a smaller number of neurons arose without PTBP1 knockdown.

FIG. 15 is a schematic of lentiviral vectors expressing shRNA for PTBP1 knockdown. shRNA includes shRNA 9 (CCA GCC CAT CTA CAT CCA GT (SEQ ID NO: 11)) and shRNA 7 (CTC AAC GTC AAG TAC AAC AA (SEQ ID NO: 12)).

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

The term “complementary” when used in connection with nucleic acid, refers to the pairing of bases, A with T or U, and G with C. The term “complementary” refers to nucleic acid molecules that are completely complementary, that is, form A to T or U pairs and G to C pairs across the entire reference sequence, as well as molecules that are partially (e.g., at least 80%, 81%, 82%, 83%, 84%, 85%, 86%, 87%, 88%, 89%, 90%, 91%, 92%, 93%, 94%, 95%, 96%, 97%, 98%, or 99%) complementary.

The terms “nucleic acid”, “nucleotide”, and “polynucleotide” encompass both DNA and RNA unless specified otherwise.

The term “polypeptide,” “peptide” or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### Methods of Generating Populations of Neurons and Producing Neurons in a Subject

A first aspect of the present disclosure relates to a method of generating a population of neurons. This method involves providing a population of human glial progenitor cells; providing a recombinant genetic construct comprising (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where the nucleic acid sequence is operably linked to the 3′ end of said promoter and/or enhancer to achieve expression of the one or more neuronal reprogramming factors; transfecting cells of the glial progenitor cell population with the recombinant genetic construct; and culturing the population after said transfecting under conditions suitable for neuron production from the transfected glial progenitor cells of the population.

As described infra, glial progenitor cells for use in the methods disclosed herein can be obtained from embryonic, fetal, or adult brain tissue, embryonic stem cells, or induced pluripotential cells. Preferably, the glial progenitor cells are isolated from ventricular and subventricular zones of the brain or from the subcortical white matter.

Glial progenitor cells can be extracted from brain tissue containing a mixed population of cells directly by using the promoter specific separation technique, as described supra. Glial specific promoters that can be used for isolating glial progenitor cells from a mixed population of cells include the CNP promoter (Scherer et al, “Differential Regulation of the 2′,3′-cyclic nucleotide 3′phosphodiesterase Gene During Oligodendrocyte Development,” Neuron 12:1363-75(1994), which is hereby incorporated by reference in its entirety), an NCAM promoter (Hoist et al., J. Biol. Chem. 269:22245-52(1994), which is hereby incorporated by reference in its entirety), a myelin basic protein promoter (Wrabetz et al., “Analysis Of The Human MBP Promoter In Primary Cultures Of Oligodendrocytes: Positive And Negative Cis-Acting Elements In The Proximal MBP Promoter Mediate Oligodendrocyte-Specific Expression Of MBP,” J. Neurosci. Res. 36:455-71(1993), which is hereby incorporated by reference in its entirety), a JC virus minimal core promoter (Krebs et al., J. Virol. 69:2434-42(1995), which is hereby incorporated by reference in its entirety), a myelin-associated glycoprotein promoter (Laszkiewicz et al., “Structural Characterization of Myelin-associated Glycoprotein Gene Core Promoter,” J. Neurosci. Res. 50(6): 928-36(1997), which is hereby incorporated by reference in its entirety), or a proteolipid protein promoter (Cook et al., “Regulation of Rodent Myelin Proteolipid Protein Gene Expression,” Neurosci. Lett. 137(1): 56-60(1992); Wight et al., “Regulation of Murine Myelin Proteolipid Protein Gene Expression,” J. Neurosci. Res. 50(6): 917-27 (1997); and Cambi et al., Neurochem. Res. 19:1055-1060 (1994), which are hereby incorporated by reference in their entirety). See also U.S. Pat. No. 6,245,564 to Goldman et al., which is hereby incorporated by reference in its entirety.

iPSCs are pluripotent cells that are derived from non-pluripotent cells, such as somatic cells. For example, and without limitation, iPSCs can be derived from tissue, peripheral blood, umbilical cord blood, and bone marrow (see, e.g., Cai et al., “Generation of Human Induced Pluripotent Stem Cells from Umbilical Cord Matrix and Amniotic Membrane Mesenchymal Cells,” J. Biol. Chem. 285 (15): 112227-11234 (2110); Giorgetti et al., “Generation of Induced Pluripotent Stem Cells from Human Cord Blood Cells with only Two Factors: Oct4 and Sox2,” Nat. Protocol. 5 (4): 811-820 (2010); Streckfuss-Bomeke et al., “Comparative Study of Human-Induced Pluripotent Stem Cells Derived from Bone Marrow Cells, Hair Keratinocytes, and Skin Fibroblasts,” Eur. Heart J. 34(33): 2618-2629 (2012); Hu et al., “Efficient Generation of Transgene-Free Induced Pluripotent Stem Cells from Normal and Neoplastic Bone Marrow and Cord Blood Mononuclear Cells,” Blood 117(14): e109-e119(2011); Sommer et al., “Generation of Human Induced Pluripotent Stem Cells from Peripheral Blood using the STEMCCA Lentiviral Vector,” J. Vis. Exp. 68:4327 (2012), which are hereby incorporated by reference in their entirety). The somatic cells are reprogrammed to an embryonic stem cell-like state using genetic manipulation. Exemplary somatic cells suitable for the formation of iPSCs include fibroblasts (see, e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. 34 (33): 2618-2629 (2012), which is hereby incorporated by reference in its entirety), such as dermal fibroblasts obtained by a skin sample or biopsy, synoviocytes from synovial tissue, keratinocytes, mature B cells, mature T cells, pancreatic β cells, melanocytes, hepatocytes, foreskin cells, cheek cells, or lung fibroblasts.

Methods of producing induced pluripotent stem cells are known in the art and typically involve expressing a combination of reprogramming factors in a somatic cell. Suitable reprogramming factors that promote and induce iPSC generation include one or more of octamer-binding transcription factor 4 (Oct4), kruppel-like factor 4 (Klf4), SRY (sex determining region Y)-box 2 (Sox2), c-Myc, Nanog, CCAAT-enhancer-binding protein alpha (C/EBPα), estrogen-related receptor beta (Esrrb), Lin28, and nuclear receptor subfamily 5, group A, member 2 (Nr5a2). In certain embodiments, at least two reprogramming factors are expressed in a somatic cell to successfully reprogram the somatic cell. In other embodiments, at least three reprogramming factors are expressed in a somatic cell to successfully reprogram the somatic cell.

iPSCs may be derived by methods known in the art, including the use integrating viral vectors (e.g., lentiviral vectors, inducible lentiviral vectors, and retroviral vectors), excisable vectors (e.g., transposon and foxed lentiviral vectors), and non-integrating vectors (e.g., adenoviral and plasmid vectors) to deliver the genes that promote cell reprogramming (Takahashi, K. and Yamanaka, S., “Induction of Pluripotent Stem Cells from Mouse Embryonic and Adult Fibroblast Cultures by Defined Factors,” Cell 126:663-676 (2006); Okita et al., “Generation of Germline-Competent Induced Pluripotent Stem Cells,” Nature 448:313-317 (2007); Nakagawa et al., Nat. Biotechnol. 26:101-106 (2007); Takahashi et al., “Induction of Pluripotent Stem Cells from Adult Human Fibroblasts by Defined Factors,” Cell 131:1-12 (2007); Meissner et al., “Direct Reprogramming of Genetically Unmodified Fibroblasts into Pluripotent Stem Cells,” Nat. Biotech. 25:1177-1181 (2007); Yu et al., “Induced Pluripotent Stem Cell Lines Derived from Human Somatic Cells,” Science 318:1917-1920 (2007); Park et al., “Reprogramming of Human Somatic Cells Pluripotency with Defined Factors,” Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication No. 2011/0200568 to Ikeda et al., U.S. Patent Application Publication No 2010/0156778 to Egusa et al., U.S. Patent Application Publication No 2012/0276070 to Musick, and U.S. Patent Application Publication No 2012/0276636 to Nakagawa, Shi et al., “Induction of Pluripotent Stem Cells from Mouse Embryonic Fibroblasts by Oct4 and Klf4 with Small-Molecule Compounds” Cell Stem Cell 3(5): 568-574 (2008), Kim et al., “Pluripotent Stem Cells Induced from Adult Neural Stem Cells by Reprogramming with Two Factors,” Nature 454:646-650 (2008), Kim et al., “Oct4-induced Pluripotency in Adult Neural Stem Cells,” Cell 136 (3): 411-419 (2009), Huangfu et al., “Induction of Pluripotent Stem Cells from Primary Human Fibroblasts with Only Oct4 and Sox2,” Nat. Biotechnol. 26:1269-1275 (2008), Zhao et al., “Two Supporting Factors Greatly Improve the Efficiency of Human iPSC Generation,” Cell Stem Cell 3:475-479 (2008), Feng et al., “Reprogramming of Fibroblasts into Induced Pluripotent Stem Cells with Orphan Nuclear Receptor Esrrb,” Nat. Cell Biol. 11:197-203 (2009), and Hanna et al., “Direct Reprogramming of Terminally Differentiated Mature B Lymphocytes to Pluripotency” Cell 133 (2): 250-264 (2008), which are hereby incorporated by reference in their entirety.

The methods of iPSC generation described above can be modified to include small molecules that enhance reprogramming efficiency or even substitute for a reprogramming factor. These small molecules include, without limitation, epigenetic modulators such as, the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as transforming growth factor beta (TGF-β) inhibitors and kinase inhibitors (e.g., kenpaullone) (see review by Sommer and Mostoslavsky, “Experimental Approaches for the Generation of Induced Pluripotent Stem Cells,” Stem Cell Res. Ther. 1:250-264 (2010), which is hereby incorporated by reference in its entirety).

Methods of obtaining highly enriched populations of glial progenitor cells from the iPSCs that are suitable for use in the methods disclosed herein are disclosed in WO2014/124087 to Goldman and Wang, and Wang et al., “Human iPSC-Derived Oligodendrocyte Progenitors Can Myelinate and Rescue a Mouse Model of Congenital Hypomyelination,” Cell Stem Cell 12 (2): 252-264 (2013), which are hereby incorporated by reference in their entirety.

In any embodiment of the methods disclosed herein, the glial progenitor cells of the population are CD140⁺, CD44⁺, or CD140⁺/CD44⁺ human glial progenitor cells.

In any embodiment of the methods disclosed herein, said culturing is effective to produce a population of medium spiny neurons, cortical interneurons, dopaminergic neurons, peripheral sensory neurons, nonadrenergic neurons, cholinergic neurons, spinal motor neurons, and/or combination thereof.

As used herein, “culturing a population of cells” refers to the maintenance of a population of cells under conditions favorable to their growth, proliferation, and/or differentiation. In any embodiment of the methods disclosed herein, said culturing a population of cells may refer to the incubation of cells in a medium in combination with soluble factors and under environmental conditions that facilitate the differentiation of the cultured cells into cells of the nervous system.

Another aspect of the present disclosure relates to a method of inducing the production of neurons in a subject in need thereof. This method involves providing a recombinant genetic construct comprising: (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where the nucleic acid sequence is operably linked to the 3′ end of the promoter and/or enhancer. This method further involves administering, to the subject in need of neuron production, the recombinant genetic construct, under conditions effective for the one or more neuronal reprogramming factors to be expressed in glial progenitor cells of the subject, thereby inducing neuron generation in said subject.

As used herein, the term “subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cat, or a dog. In some embodiments, the subject is a research animal. In some embodiments, the subject is genetically engineered, e.g., a genetically engineered non-human subject.

The subject may be an adult subject. In some embodiments, the subject is at least 18 years old, at least 20 years old, at least 25 years old, at least 30 years old, at least 35 years old, at least 40 years old, at least 45 years old, at least 50 years old, at least 55 years old, at least 60 years old, at least 65 years old, at least 70 years old, at least 75 years old, at least 80 years old, at least 85 years old, at least 90 years old, at least 95 years old, at least 100 years old, or more. In some embodiments, the adult subject is between 18 to 100 years old, 20 to 100 years old, 30 to 100 years old, 40 to 100 years old, 50 to 100 years old, 50 to 100 years old, 60 to 100 years old, 70 to 100 years old, 80 to 100 years old, or 90 to 100 years old.

In any embodiment of the methods according to the present disclosure, the subject has a neurodegenerative condition or disease. As described herein, the term “neurodegenerative condition or disease” refers to a chronic progressive neuropathy characterized by selective and generally symmetrical loss of neurons in motor, sensory, or cognitive systems. The neurodegenerative condition or disease may be selected from, e.g., the group consisting of Huntington's disease, Alzheimer's disease, frontotemporal dementia, Parkinson's disease, multisystem atrophy, and amyotrophic lateral sclerosis, and conditions mediated by a deficiency in myelin.

Huntington's disease is an autosomal dominant neurodegenerative disease characterized by a relentlessly progressive movement disorder with devastating psychiatric and cognitive deterioration. Huntington's disease is associated with a consistent and severe atrophy of the neostriatum which is related to a marked loss of the GABAergic medium-sized spiny projection neurons, the major output neurons of the striatum. In some embodiments, the subject has Huntington's disease.

Alzheimer's disease is a neurodegenerative disease with insidious onset and progressive impairment of behavioral and cognitive functions including memory, comprehension, language, attention, reasoning, and judgment. Alzheimer's disease is caused by neuronal cell death. It typically starts in the entorhinal cortex in the hippocampus. In some embodiments, the subject has Alzheimer's disease.

Frontotemporal dementia is a group of related conditions resulting from the progressive degeneration of the temporal and frontal lobes of the brain. These areas of the brain play a significant role in decision-making, behavioral control, emotion, and language. In some embodiments, the subject has frontotemporal dementia.

Parkinson's disease is a progressive nervous system disorder that affects movement and which is characterized by progressive neurodegeneration. In some embodiments, the subject has Parkinson's disease.

Multisystem atrophy is a progressive neurodegenerative disorder characterized by a combination of symptoms that affect both the autonomic nervous system (the part of the nervous system that controls involuntary action such as blood pressure or digestion) and movement. The symptoms reflect the progressive loss of function and death of different types of nerve cells in the brain and spinal cord. In some embodiments, the subject has multisystem atrophy.

Amyotrophic lateral sclerosis (ALS, commonly called “Lou Gehrig's disease”) is the most common motor neuron disease in adults. Motor neuron diseases are neurodegenerative diseases that cause selective loss of the nerve cells that directly connect the brain to muscles. In some embodiments, the subject has amyotrophic lateral sclerosis.

The condition mediated by a deficiency in myelin may be a leukodystrophy or a white matter disease. In some embodiments, the condition mediated by a deficiency in myelin may be selected from the group consisting of pediatric leukodystrophies, the lysosomal storage diseases, congenital dysmyelination, cerebral palsy, inflammatory demyelination, post-infectious and post-vaccinial leukoencephalitis, radiation- or chemotherapy induced demyelination, and vascular demyelination.

Leukodystrophy refers to a group of rare, primarily inherited neurological disorders known as the leukodystrophies that result from the abnormal production, processing, or development of myelin and other components of central nervous system (CNS) white matter, such as cells called oligodendrocytes and astrocytes. All leukodystrophies are the result of genetic defects (mutations).

In some embodiments, the condition mediated by a deficiency in myelin requires myelination. In other embodiments, the condition mediated by a deficiency in myelin requires remyelination. In some embodiments, the condition requiring remyelination is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, and cerebral palsy.

In any embodiment of the methods according to the present disclosure, the subject has a neurodegenerative condition or disease. Exemplary neuropsychiatric diseases or neuropsychiatric disorders include, without limitation, schizophrenia, autism spectrum disorder, and bipolar disorder.

Schizophrenia is a serious mental illness that affects how a person thinks, feels, and behaves. The symptoms of schizophrenia generally fall into the following three categories: 1) psychotic symptoms including altered perceptions, 2) negative symptoms including loss of motivation, disinterest and lack of enjoyment, and 3) cognitive symptoms including problems in attention, concentration, and memory.

Autism spectrum disorder is a neurodevelopment disorder that causes a wide range of impairments in social communication and restricted and repetitive behaviors.

Bipolar disorder is a serious mental illness characterized by extreme mood swings. They can include extreme excitement episodes or extreme depressive feelings. Three types of bipolar disorder include: 1) Bipolar I Disorder, defined by manic episodes, 2) Bipolar II Disorder, that is defined by depressive episodes, and 3) Cyclothymic Disorder, defined by periods of hypomanic and depressive symptoms.

Suitable recombinant genetic constructs for use in the methods according to the present disclosure are described in detail infra.

In some embodiments, administering the recombinant genetic construct of the present disclosure is effective to treat the neurological condition or disease in the subject. In accordance with such embodiments, “treating” the disease or disorder encompasses: (1) preventing, delaying, or reducing the incidence and/or likelihood of the appearance of at least one clinical or sub-clinical symptom of the state, disorder, or disease developing in a subject that may be afflicted with or predisposed to the state, disorder, or disease, but does not yet experience or display clinical or subclinical symptoms of the state, disorder, or disease; or (2) inhibiting the state, disorder, or disease, i.e., arresting, reducing or delaying the development of the disease or a relapse thereof or at least one clinical or sub-clinical symptom thereof; or (3) relieving the disease, i.e., causing regression of the state, disorder, or disease or at least one of its clinical or sub-clinical symptoms. The benefit to a subject to be treated is either statistically significant or at least perceptible to the subject or to the physician.

Suitable methods of administering the recombinant genetic construct to a subject are well known in the art.

In some embodiments, the recombinant genetic construct is administered to one or more sites of the brain, the brain stem, the spinal cord, or a combination thereof.

In some embodiments, the preparation is administered intraventricularly, intracallosally, or intraparenchymally.

Delivery of the recombinant genetic construct to the subject can include either a single step or a multiple step injection directly into the nervous system. Multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

### Recombinant Genetic Constructs

Applicant has found that human glial progenitor cells are an appropriate and effective cellular substrate for induced neuronal phenoconversion, and have designed recombinant genetic constructs and expression vectors that achieve the efficient, cell-specific targeting of human glial progenitor cells, permitting the specific transduction of human glial progenitor cells with neuronal reprogramming factors that mediate reprogramming to neuronal phenotype. Accordingly, another aspect of the present disclosure relates to a recombinant genetic construct comprising: (i) a promoter and/or enhancer for a gene which is selectively or specifically expressed by human glial progenitor cells, and (ii) a nucleic acid sequence encoding one or more neuronal reprogramming factors for producing neurons from glial progenitor cells, where said nucleic acid sequence is operably linked to the 3′ end of said promoter and/or enhancer to achieve expression of the one or more neuronal programming factors in glial progenitor cells.

The “recombinant genetic constructs” of the disclosure are nucleic acid molecules containing a combination of two or more genetic elements not naturally occurring together. As described in more detail infra, the recombinant genetic construct may be expressed in a population of glial progenitor cells to induce neurogenesis in the population of glial progenitor cells.

A “regulatory element” is a nucleic acid molecule that can influence the expression of an operably linked coding sequence in a particular host organism. The term “regulatory element” is used broadly to cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873, which is hereby incorporated by reference in its entirety). Regulatory elements include, without limitation, transcriptional and translational control sequences, such as promoters, enhancers, splicing signals, polyadenylation signals, terminators, protein degradation signals, 2A sequences, and the like, that provide for and/or regulate expression of a coding sequence and/or production of an encoded polypeptide in a host cell.

The term “promoter” refers to a nucleotide sequence that permits binding of RNA polymerase and directs the transcription of a gene or a nucleic acid molecule encoding one or more neuronal reprogramming factors of interest. Typically, a promoter is located in the 5′ non-coding region of a gene, proximal to the transcriptional start site of a gene or nucleic acid sequence of interest. Sequence elements within promoters that function in the initiation of transcription are often characterized by consensus nucleotide sequences.

The term “enhancer” refers to a segment of DNA which contains sequences capable of providing enhanced transcription and in some instances can function independent of their orientation relative to another control sequence. Enhancers are cis-acting DNA sequences that can function cooperatively or additively with promoters and/or other enhancer elements. The term “promoter/enhancer” or “promoter-inclusive regulatory element” refers to a segment of DNA which contains sequences capable of providing both promoter and enhancer functions.

As described herein, the nucleic acid sequence encoding one or more neuronal reprogramming factors according to the present disclosure is operably linked to the promoter and/or enhancer according to the present disclosure to achieve expression of the one or more neuronal programming factors in glial progenitor cells. The term “operably linked” describes the connection between regulatory elements (e.g., a promoter and/or enhancer according to the present disclosure) and a nucleic acid sequence encoding a sequence of interest (e.g., a nucleic acid sequence encoding one or more neuronal reprogramming factors according to the present disclosure). Typically, expression of the nucleic acid sequence encoding a sequence of interest is placed under the control of one or more regulatory elements, for example, without limitation, a promoter and/or an enhancer. A nucleic acid sequence encoding a sequence of interest is said to be “operably linked to” the regulatory elements, meaning that the transcription of the nucleic acid sequence encoding the sequence of interest is controlled or influenced by the regulatory element(s). For instance, a promoter and/or enhancer for a gene which is selectively or specifically expressed by human glial progenitor cells is operably linked to a nucleic acid sequence encoding the one or more neuronal reprogramming factors if the promoter effects transcription or expression of the nucleic acid sequence encoding the one or more neural reprogramming factors.

In any embodiment of the methods or recombinant genetic constructs disclosed herein, the recombinant genetic construct comprises a promoter and/or enhancer for a gene which is selectively or specifically expressed by human glial progenitor cells (e.g., a platelet derived growth factor alpha (PDGFRA) promoter, a zinc finger protein 488 (ZNF488) promoter, a G protein-coupled receptor (GPR17) promoter, an oligodendrocyte Transcription Factor 2 (OLIG2) promoter, a chondroitin sulfate proteoglycan 4 (CSPG4) promoter, and a SRY-box transcription factor 10 (SOX10) promoter, sequences of which are identified in Table 1 supra.

In accordance with such embodiments of the present disclosure, the gene selectively or specifically expressed by human glial progenitor cells is selected from the group consisting of platelet derived growth factor alpha (PDGFRA), zinc finger protein 488 (ZNF488), G protein-coupled receptor (GPR17), oligodendrocyte Transcription Factor 2 (OLIG2), chondroitin sulfate proteoglycan 4 (CSPG4), and SRY-box transcription factor 10 (SOX10), sequences of which are identified in Table 1 below.

TABLE 1
Exemplary Genes Selectively or Specifically Expressed by Glial Progenitor Cells

| | | NCBI Gene | NCBI Reference |
|---|---|---|---|
| Gene | Organism | ID No.* | Sequence:* |
| Platelet derived growth factor alpha (PDGFRA) | Homo sapiens | 5156 | NG_009250.1 |
| Zinc finger protein 488 (ZNF488) | Homo sapiens | 118738 | |
| G protein-coupled receptor (GPR17) | Homo sapiens | 2840 | NG_042235.1 |
| Oligodendrocyte Transcription Factor 2 | Homo sapiens | 10215 | NG_011834.1 |
| (OLIG2) | | | |
| chondroitin sulfate proteoglycan 4 (CSPG4) | Homo sapiens | 1464 | |
| SRY-box transcription factor 10 (SOX10) | Homo sapiens | 6663 | NG_007948.1 |

*Each of which is hereby incorporated by reference in its entirety.

In some embodiments of the methods and recombinant genetic constructs according to the present disclosure, the gene selectively or specifically expressed by human glial progenitor cells is GPR17. In accordance with such embodiments, the promoter and/or enhancer for a gene which is selectively or specifically expressed by human glial progenitor cells according to the present disclosure may be a GPR17 promoter-inclusive regulatory element. As used herein, the phrase “GPR17 promoter-inclusive regulatory element” refers to a nucleotide sequence that directs the glial progenitor cell-specific transcription of a nucleic acid sequence encoding a sequence of interest (e.g., a nucleic acid sequence encoding one or more neuronal reprogramming factors according to the present disclosure). The GPR17 promoter-inclusive regulatory element according to the present disclosure may comprise a portion of the 5′ untranslated region of GPR17 (NCBI Gene ID: 2840, which is hereby incorporated by reference in its entirety).

In some embodiments of the methods and recombinant genetic constructs according to the present disclosure, the GPR17 promoter-inclusive regulatory element may comprise the nucleic acid sequence of SEQ ID NO: 1 below. GGATACGGAAGAGATCCAATCGCAGACCCAAGATCCCCACCCAGGTTATGGTGGGCAGACCCCA GATGCCAGGGCCACCCATTCAGCATCCCTCCCTGGACCCCAGGACCTGCTACTGCTGGGTGTCT GGACTCCATCCTGCACAGCACTGTGCTCCATCTGCCCTGGGGTGTCTCATCATCAGCTGTGTGC AGGGCAAGGGGCCCAAACAAAGGCCCAGCAGTCACTGGCTAAGCTGCCGACTGGCTCTCTGTGC CTCCCCAAGACCCTATGTGCCCAGCAGGGGGCAACAGCTCAGGGTCAGCTGACCGAATGCCTCG GTGAATGAATGACTCTACAAGAGAGGAAGGGAGCCTCGGTGGGCATCATCTCCCCTCGACTACT GGCCAGAGCCCTGGCTCTTACACCCCAGCGACGGGAAGCAGTTGTGGCCTGTGGCTTCAGTCTT CATCACCACAATCCCTGAAGCCCACCCTTGCCCAGACACCTGTGCCCCAGCCCCAACCCCAGGC CACCTCCTCAGCAGGTCTGGGGCTGAGCTGCCCCACCTGGCGCCTATGGCGGCCAGCCCATGCC CCCTGCGGTGCCTCTGTCCCAGACTCAGCATGTAGGCCCCATGACCCCACTCCACATTCTGGTG ACTCCTCCTGAGCGTCAGGACAACACTCAACCCACGAGGAATTATTTCTGTCTCAAAGATGCAG GAATCAGCTCAACGCCTCAAAACTCCATCACCACGGTCAATGCCCTTGAAGCCATCGACAGTGA TCACCCCAATAACAGAAGGTCTGTGAGCCCAGAAATGCCCTGCTCAGGGTGGTTAGCTTCAAGC CACCACCTTTCCAACCAGCCTGGGCCAGTTCTTCCAGACAGCCGCCTGCGGGCACAACAGGAAA GAGACCTGCGCCCCGGCTCAGACACCTCACACCCAGCTGGCTCTCAGGCCAGACAAACTGGGAA GCCCATCTCTCTTGAAGGAAGTCCAGATGGGAAACAGCTTCTCAACAGACCAGATCACAGCATC AGATCTAAAGGTGGCCTTCAGAATTCTTTTTCAGGTTGAATTAGGATCAAATCTAAGAATTCTA AATTCAAAATGCAGCAGAAAAACAAAACACACACACACACGGAGCCTAAGTTCTGGAGTGACAT GTGCTTGGGTTCAAATCCTGGCTCTGTTGCTTCCTACTGTTTGTTGATGGGTGAGTTTCTTCAT TTGCCTGAGCCTCAGTTTCCTTGTCTGTAAAATGGGGCAATAATCCCAGCTGCACAGGGTGATG TGAAGAGACAAATTTAAGACACTGCCCCTTAAATGCTAGCCACATACATACAGTTTTCAATGTT TAAACAACAAAATGTAAAGTCTTTTGAAACCAGGAAGGGTGATTTGGTTTCCCATGTTGCTGGA TGTATCATTTTCAGAAAGACAGAGAGAAATGAACTTTGTTCACTCAGTCTCAGAGGCGGCCGCC GGCAGCATTCAAAGGCACCCCAGCCCGGAGCCACCCCAGGGAGGAGCCCCAGGCCAGCGGTCAG ATTCATGGGCTTCCGTGCAGAAGGGGAGCTGCACCGGCGAGCACCCGGCCTCTGAGCTGAGCCG CATCCTCACGGACAGGACAGCGCCCCATTATGAGGCTCCTGCAGCTGTTCCTCGCTCCAGATAA AGGCCATGATTTATTCTGTGTGCCCAAATGGGGCCTCATTATACAGGGCAGGACACAAGGACCC TACAGCAAGTGTCCTCAAAGAGTCGCCTCTCACTCCGTGAGCAAGACTCCTCGGCCTCCCACCC TCCGTTCACAGGCCCCCTCCGCCGTCTGCGGGCGCAGGCCTGGGAGCGCCGCCTGTTGCCATGA CAGCCGGCCCCTCCCTGCCCCCCATCAGTAGGAAATCATCCCCTTCTGAAACGTCCTGTTGTGT CCCTCAGCTCCAGCCCAAGCCCCCCACCCAGCCCCCGCCTGCTCTGAGTCTCTGAGACAGTCAC ACACTCAGACTATGTGGCCAAGCTGGGGGGGGGGGGCATGGGCTAGGGACACACTAGAATATTC ACGCTCCGGTGGCAGCAGCAGCAGCAGCCAGAGGAGCAGCCCGACACAACAAGGGACCCCTCAG GAATGAAGCAGCCTTTCAGGGCCAGAGGGGCTGTGGTCTCCCTTCCTCTCCTTAAATAGCCAGC GTTCCACCCACAGCGGCAAGGGC (SEQ ID NO: 1). Thus, in some embodiments of the methods and recombinant genetic constructs according to the present disclosure, the GPR17 promoter-inclusive regulatory element has the sequence of SEQ ID NO: 1.

In some embodiments, the GPR17 promoter-inclusive regulatory element comprises a portion of SEQ ID NO: 1. For example, the GPR17 promoter-inclusive regulatory element may comprise a continuous stretch of 1-100, 1-200, 1-300, 1-400, 1-500, 1-600, 1-700, 1-800, 1-900, 1-1,000, 1-1,100, 1-1,200, 1-1,300, 1-1,400, 1-1,500, 1-1,600, 1-1,700, 1-1,800, 1-1,900, 1-2,000, 1-2, 100, or 1-2,198 of SEQ ID NO: 1.

In some embodiments of the methods and recombinant genetic constructs according to the present disclosure, the GPR17 promoter-inclusive regulatory element comprises a 5′ untranslated region of Homo sapiens G protein-coupled receptor 17 (GPR17) comprising the nucleic acid sequence of SEQ ID NO: 2 below. GTTGCTGGATGTATCATTTTCAGAAAGACAGAGAGAAATGAACTTTGTTCACTCAGTCTCAGAG GCGGCCGCCGGCAGCATTCAAAGGCACCCCAGCCCGGAGCCACCCCAGGGAGGAGCCCCAGGCC AGCGGTCAGATTCATGGGCTTCCGTGCAGAAGGGGAGCTGCACCGGCGAGCACCCGGCCTCTGA GCTGAGCCGCATCCTCACGGACAGGACAGCGCCCCATTATGAGGCTCCTGCAGCTGTTCCTCGC TCCAGATAAAGGCCATGATTTATTCTGTGTGCCCAAATGGGGCCTCATTATACAGGGCAGGACA CAAGGACCCTACAGCAAGTGTCCTCAAAGAGTCGCCTCTCACTCCGTGAGCAAGACTCCTCGGC CTCCCACCCTCCGTTCACAGGCCCCCTCCGCCGTCTGCGGGCGCAGGCCTGGGAGCGCCGCCTG TTGCCATGACAGCCGGCCCCTCCCTGCCCCCCATCAGTAGGAAATCATCCCCTTCTGAAACGTC CTGTTGTGTCCCTCAGCTCCAGCCCAAGCCCCCCACCCAGCCCCCGCCTGCTCTGAGTCTCTGA GACAGTCACACACTCAGACTATGTGGCCAAGCTGGGGGGGGGGGGCATGGGCTAGGGACACACT AGAATATTCACGCTCCGGTGGCAGCAGCAGCAGCAGCCAGAGGAGCAGCCCGACACAACAAGGG ACCCCTCAGGAATGAAGCAGCCTTTCAGGGCCAGAGGGGCTGTGGTCTCCCTTCCTCTCCTTAA ATAGCCAGCGTTCCACCCACAGCGGCAAGGG (SEQ ID NO: 2; GPR17 (0.8)). Thus, in some embodiments of the methods and recombinant genetic constructs according to the present disclosure, the GPR17 promoter-inclusive regulatory element has the sequence of SEQ ID NO: 2.

In some embodiments, the GPR17 promoter-inclusive regulatory element comprises a modified nucleic acid sequence of SEQ ID NO: 1 or SEQ ID NO: 2. The modified sequence may have at least 80% sequence identity to the nucleic acid sequence of SEQ ID NO: 1 or SEQ ID NO: 2. In some embodiments, the modified sequence has 81%, 82%, 83%, 84%, 85%, 86%, 87%, 88%, 89%, 90%, 91%, 92%, 93%, 94%, 95%, 96%, 97%, 98%, 99%, or more sequence identity with the nucleic acid sequence of SEQ ID NO: 1 or SEQ ID NO: 2. In some embodiments, the modified sequence contains a mutation that enhances transcription of the nucleic acid sequence encoding an effector molecule. In some embodiments of the methods and recombinant genetic constructs according to the present disclosure, the GPR17 promoter-inclusive regulatory element has the sequence of SEQ ID NO: 1 or SEQ ID NO: 2.

In some embodiments of the methods and recombinant genetic constructs according to the present disclosure, the one or more neuronal reprogramming factors are selected from the group consisting of medium spiny neuron reprogramming factors, cortical interneuron reprogramming factors, dopaminergic neuron reprogramming factors, peripheral sensory neuron reprogramming factors, nonadrenergic neuronal reprogramming factors, cholinergic reprogramming factors, and spinal motor neuron reprogramming factors.

In any embodiment of the methods and recombinant genetic constructs according to the present disclosure, the one or more reprogramming factors is a miRNA. As used herein, the term “microRNA” or “miRNA” refers to a class of small RNA molecules that may negatively regulate gene expression (see, e.g., Lam et al., “siRNA Versus miRNA as Therapeutics for Gene Silencing,” Mol. Ther. Nucleic Acids 4(9): e252 (2015), which is hereby incorporated by reference in its entirety). miRNA gene transcription is carried out by RNA polymerase II in the nucleus to give primary miRNA (pri-miRNA), which is a 5′ capped, 3′ polyadenylated RNA with double-stranded stem-loop structure. The pri-miRNA is then cleaved by a microprocessor complex (comprising Drosha and microprocessor complex subunit DCGR8) to form precursor miRNA (pre-miRNA), which is a duplex that contains 70-100 nucleotides with interspersed mismatches and adopts a loop structure. The pre-miRNA is subsequently transported by Exportin 5 from the nucleus to the cytoplasm, where it is further processed by Dicer into a miRNA duplex of 18-25 nucleotides. The miRNA duplex then associates with the RISC forming a complex called miRISC. The miRNA duplex is unwound, releasing and discarding the passenger strand (sense strand). The mature single-stranded miRNA guides the miRISC to the target mRNAs. Mature miRNA may bind to a target mRNA through partial complementary base pairing with the consequence that the target gene silencing occurs via translational repression, degradation, and/or cleavage.

miRNAs suitable for use in the recombinant genetic constructs disclosed herein include, without limitation, hsa-mir-9 (hsa-mir-9-5p) (miRBase Accession No. MIMAT0000441, which is hereby incorporated by reference in its entirety); hsa-mir-9 (hsa-mir-9-3p) (miRBase Accession No. MIMAT0000442, which is hereby incorporated by reference in its entirety); hsa-mir-124-1 (miRBase Accession No. MI0000443, which is hereby incorporated by reference in its entirety); hsa-mir-124-2 (miRBase Accession No. MI0000444, which is hereby incorporated by reference in its entirety); and hsa-mir-124-3 (miRBase Accession No. MI0000445, which is hereby incorporated by reference in its entirety) (see, e.g., Nowek et al., “The Versatile Nature of miR-9/9* in Human Cancer,” Oncotarget. 9(29): 20838-20854 (2018), which is hereby incorporated by reference in its entirety).

miR-9/9* and miR-124 are essential for neuronal differentiation and the maintenance of neuronal identity through the repression of anti-neural genes including cofactors of the REST complex, RCOR1, and SCP1 (see, e.g., Lu and Yoo, “Mechanistic Insights Into MicroRNA-Induced Neuronal Reprogramming of Human Adult Fibroblasts,” Front. Neurosci. 12:522 (2018), which is hereby incorporated by reference in its entirety). miR-9 (miR-9-5p) and miR-9* (miR-9-3p) are two miRNAs that originate from the same precursor and are highly conserved during evolution from flies to humans (see Nowek et al., “The Versatile Nature of miR-9/9* in Human Cancer,” Oncotarget 9 (29): 20838-20854 (2018), which is hereby incorporated by reference in its entirety). miR-124 represses translation of a large number of non-neuronal transcripts (Lim et al., “Microarray Analysis shows that some microRNAs Downregulate Large Numbers of Target mRNAs,” Nature 43:769-773 (2005), which is hereby incorporated by reference in its entirety) and is a well-known regulator of the transcription silencing complex built on REST, which represses a large array of neuronal-specific genes in non-neuronal cells; this include miR-124 itself, thus forming an auto-regulatory loop during neuronal differentiation (Xue et al., “Direct Conversion of Fibroblasts to Neurons by Reprogramming PTB-Regulated microRNA Circuits,” (′e//152 (1-2): 82-96 (2013), which is hereby incorporated by reference in its entirety).

In some embodiments of the methods and recombinant genetic constructs according to the present disclosure, the one or more reprogramming factors is selected from, e.g., miR-9/9* and miR-124.

In some embodiments of the methods and recombinant genetic constructs according to the present disclosure, the one or more reprogramming factors is MIR124/9 having the sequence of SEQ ID NO: 8 below.

(SEQ ID NO: 8)
CCTCTGGCTCTCCGCGTGCGCCCGGGGCTGCGGGAGCGGAGTCCGCTCAGGCCCCCGCGCCGGG
TCGGCGGCGGCACGTGGGGCCCACAGCGCGGCAGCGGTGCCGGCGAATGGGAGGCCCGTTTCTC
TCTTTGGTTATCTAGCTGTATGAGTGCCACAGAGCCGTCATAAAGCTAGATAACCGAAAGTAGA
AATGACTCTCACAACTTCTGCGTGCGATGGCCCGGCCGGCCGCCCTGGCCACAGGAGCCAGAGG
AGCCCAGAGAAGCCCACGCCACCCAGGGATTCCAGCTCGACGGAGCGGCCACGCCGGGACCAGG
TATCCGACCTCGGCTCTCCTTCTAGTAGGTGGGAGTACTGCTCAGAGCTACAACTCTAGGAGTA
GGGACTCCAAGCCTAGAGCTCCAAGAGAGGGTGAAGGGCAGGGAGAAAATTATAGTAATAGTTG
CAATGAGTCACTTGCTTCTAGATCAAGATCAGAGACTCTGCTCTCCGTGTTCACAGCGGACCTT
GATTTAATGTCATACAATTAAGGCACGCGGTGAATGCCAAGAGCGG

As described herein, regulated RNA processing plays a critical role in neuronal differentiation. The polypyrimidine tract binding protein PTB and its homolog nPTB undergo a programmed switch during neuronal differentiation. miR-124 is able to modulate such switch by reducing PTB, thereby reprogramming an array of neuronal-specific alternative splicing events and forced expression of PTB is able to block miR-124 induced neuronal differentiation (Xue et al., “Direct Conversion of Fibroblasts to Neurons by Reprogramming PTB-Regulated microRNA Circuits,” Cell 152 (1-2): 82-96 (2013), which is hereby incorporated by reference in its entirety). Thus, in some embodiments of the methods and recombinant genetic constructs according to the present disclosure, the one or more neuronal reprogramming factors is an inhibitor of polypyrimidine-tract-binding protein 1 (PTBP1; PTB).

The inhibitor may be a nucleic acid molecule inhibitor. As used herein, the term “nucleic acid inhibitor” refers to a nucleic acid that reduces or eliminates the expression of a target gene (e.g., PTB). The nucleic acid inhibitor typically contains a region that specifically targets a sequence in the target gene or target gene mRNA to achieve target-specific inhibition. Typically, the targeting region of the nucleic acid inhibitor comprises a sequence that is sufficiently complementary to a sequence on the target gene or target gene mRNA to direct the effect of the nucleic acid inhibitor to the specified target gene or target gene mRNA. For example, a “nucleic acid inhibitor of PTB” reduces or eliminates the expression of a PTB gene.

As used herein, the term “reduce” or “reduces” refers to its meaning as is generally accepted in the art. With reference to exemplary nucleic acid inhibitors (e.g., nucleic acid inhibitors of PTBP1 selected from a miRNA, a shRNAi, a siRNA, and an antisense oligonucleotide), “reduce” or “reduces” generally refers to a suppression in the transcription and/or translation of a gene (e.g., PTBP1) or in the levels of the gene product relative to the transcription and/or translation of the gene observed in the absence of the nucleic acid inhibitor. In some embodiments, the reduction in the transcription and/or translation of a gene or in the levels of the gene product is at least 10%, at least 20%, at least 30%, at least 40%, at least 50%, at least 60%, at least 70%, at least 80%, at least 90%, at least 95%, at least 96%, at least 97%, at least 98%, at least 99%, up to 100% (i.e., no detectable transcription and/or translation) or a reduction of at least 2-fold, at least 5-fold, at least 10-fold, at least 20-fold, at least 30-fold, at least 40-fold, at least 50-fold, or more relative to that observed in the absence of the nucleic acid inhibitor molecule according to the present disclosure.

In some embodiments of the methods and recombinant genetic constructs according to the present disclosure, the inhibitor mediates short hairpin RNA interference of a selected target (e.g., PTBP1). As used herein, the term “short hairpin RNA interference” or “shRNAi” is mediated by a class of small RNA molecules that negatively regulate gene expression. Short hairpin RNA (shRNA) molecules comprise the sense and antisense sequences from a target gene connected by a loop. Once transcribed, shRNA molecules are transported from the nucleus into the cytoplasm where the enzyme Dicer processes them into small/short interfering RNAs (siRNAs). The term “short interfering RNA” or “siRNA” refers to short nucleic acid molecules typically 21-23 nucleotides in length with 3′-two nucleotide overhangs (see, e.g., McManus & Sharp, “Gene Silencing in Mammals by Small Interfering RNAs,” Nat. Rev. Genet. 3(10): 737-747 (2002), which is hereby incorporated by reference in its entirety). siRNA interacts with and activates the RNA-induced silencing complex (“RISC”). The endonuclease argonaute 2 (AGO2) component of the RISC cleaves the passenger strand (sense strand) of the siRNA while the guide strand (antisense strand) remains associated with the RISC. Subsequently, the guide strand guides the active RISC to its target mRNA for cleavage by AGO2. As the guide strand only binds to mRNA that is fully complementary to it, siRNA causes specific gene silencing (see, e.g., Lam et al., “siRNA Versus miRNA as Therapeutics for Gene Silencing,” Mol. Ther. Nucleic Acids 4(9): e252 (2015), which is hereby incorporated by reference in its entirety).

In some embodiments of the methods and recombinant genetic constructs according to the present disclosure, the nucleic acid inhibitor of PTB comprises a PTBP1 siRNA and/or a PTBP1 shRNA.

In some embodiments of the methods and recombinant genetic constructs according to the present disclosure, the nucleic acid inhibitor of PTB comprises one or more shRNA having the sequence of shRNA6 (SEQ ID NO: 3), shRNA8 (SEQ ID NO: 4), shRNA9 (SEQ ID NO: 5), shRNA 9 (SEQ ID NO: 11), and/or shRNA 7 (SEQ ID NO: 12) below.

(SEQ ID NO: 3)
CCAAAGCCTCTTTATTCTCTT
(SEQ ID NO: 4)
CACTATGGTTAACTACTATAC
(SEQ ID NO: 5)
GGGTGAAGATCCTGTTCAATA
SEQ ID NO: 11)
CCAGCCCATCTACATCCAGT
(SEQ ID NO: 12)
CTCAACGTCAAGTACAACAA

Methods of designing nucleic acid inhibitors are well known in the art and suitable for designing nucleic acid inhibitors for use in the recombinant genetic constructs described herein (see, e.g., Lam et al., “siRNA Versus miRNA as Therapeutics for Gene Silencing,” Mol. Ther. Nucleic Acids 4(9): e252 (2015) and Kulkarni et al., “The Current Landscape of Nucleic Acid Therapeutics,” Nature Nanotechnology 16:630-643 (2021), which are hereby incorporated by reference in their entirety).

Nucleic acid inhibitors are designed to target, e.g., PTBP1, in a sequence specific manner. The sequence of PTBP1 is well known in the art and accessible via various curated databases, e.g., NCBI nucleotide or gene database.

In some embodiments, the PTBP1 siRNA or PTBP1 shRNA is designed to target the sequence of PTBP1 transcript variant X1 mRNA (NCBI Reference Sequence: XM_005259597.2, which is hereby incorporated by reference in its entirety), or a portion thereof.

The inhibitor of PTBP1 may be a nuclease-based gene editing system. As used herein, the term “nuclease-based gene editing system” refers to a system comprising a nuclease or a derivative thereof that can be recruited to a target sequence in the genome. The system may comprise a Clustered Regularly Interspaced Short Palindromic Repeat-associated (“Cas”) protein (e.g., Cas9, Cas12a, and Cas12b), a zinc finger nuclease (“ZFNs”), or a transcription activator-like effector nuclease (“TALEN”).

As described herein, Cas proteins form a ribonucleoprotein complex with a guide RNA, which guides the Cas protein to a target DNA sequence. Suitable Cas proteins include Cas nucleases (i.e., Cas proteins capable of introducing a double strand break at a target nucleic acid sequence), Cas nickases (i.e., Cas protein derivatives capable of introducing a single strand break at a target nucleic acid sequence), and nuclease dead Cas (dCas) proteins (i.e., Cas protein derivatives that do not have any nuclease activity).

In some embodiments, the Cas protein is a Cas9 protein. As used herein, the term “Cas9 protein” or “Cas9” includes any of the recombinant or naturally occurring forms of the CRISPR-associated protein 9 (Cas9) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas9 protein. In some embodiments, the Cas9 protein is substantially identical to the protein identified by the UniProt reference number Q99ZW2, G3ECR1, J7RUA5, AOQ5Y3, or J3F2B0 (which are hereby incorporated by reference in their entirety) or a variant or homolog having substantial identity thereto. In some embodiments, the Cas9 protein is selected from the group consisting of a Cas9 nuclease, a Cas9 nickases, and a nuclease dead Cas 9 (“dCas9”).

In some embodiments, the Cas protein is a Cas12a protein. As used herein, the term “Cas12a protein” or “Cas12a” includes any of the recombinant or naturally-occurring forms of the CRISPR-associated protein 12 (Cas12a) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas12a protein. In some embodiments, the Cas12a protein is substantially identical to the protein identified by the UniProt reference number AOQ7Q2, U2UMQ6, A0A7C6JPC1, A0A7C9HOZ9, or A0A7JOAY55 (which are hereby incorporated by reference in their entirety) or a variant or homolog having substantial identity thereto. In some embodiments, the Cas 12a protein is selected from the group consisting of a Cas12a nuclease, a Cas12a nickase, and a nuclease dead Cas12a (“dCas12a”).

In some embodiments, the Cas protein is a Cas12b protein. As used herein, the term “Cas12b protein” or “Cas12b” includes any of the recombinant or naturally-occurring forms of the CRISPR-associated protein 12 (Cas12b) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas12b protein. In some embodiments, the Cas12b protein is substantially identical to the protein identified by the UniProt reference number TOD7A2, A0A613SPI6, A0A6I7FUC4, A0A6N9TP17, A0A6MIUF64, A0A7Y8V748, A0A7X7KIS4, A0A7X8X2U5, or A0A7X8UMW7 (which are hereby incorporated by reference in their entirety) or a variant or homolog having substantial identity thereto. In some embodiments, the Cas 12b protein is selected from the group consisting of a Cas12b nuclease, a Cas12b nickase, and a nuclease dead Cas12b (“dCas12b”).

As used herein, the term “guide RNA” or “gRNA” refers to a ribonucleotide sequence capable of binding a nucleoprotein, thereby forming ribonucleoprotein complex. In accordance with the methods and systems of the present disclosure, the guide RNA comprises (i) a DNA-targeting sequence that is complementary to a target nucleic acid sequence of ZNF274, MAX, E2F6, IKZI3, or STAT3 sequence and (ii) a binding sequence for the Cas protein (e.g., Cas9 nuclease, Cas9 nickase, dCas9, Cas12a nuclease, Cas12a nickase, or dCas12a).

In some embodiments of the methods and recombinant genetic constructs according to the present disclosure, the guide RNA is a single guide RNA molecule (single RNA nucleic acid), which may include a “single-guide RNA” or “sgRNA”. In other embodiments of the methods and recombinant genetic constructs according to the present disclosure, the nucleic acid of the present disclosure includes two RNA molecules (e.g., joined together via hybridization at the binding sequence). Thus, the term guide RNA is inclusive, referring both to two-molecule nucleic acids and to single molecule nucleic acids (e.g., sgRNAs).

In some embodiments of the methods and recombinant genetic constructs according to the present disclosure, the gRNA is 10, 20, 30, 40, 50, 60, 70, 80, 90, 100 or more nucleic acid residues in length. In some embodiments, the gRNA is from 10 to 30 nucleic acid residues in length. In some embodiments, the gRNA is 20 nucleic acid residues in length. In some embodiments, the length of the gRNA is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In some embodiments, the gRNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In some embodiments, the gRNA is from 10 to 15, 10 to 20, 10 to 30, 10 to 40, or 10 to 50 residues in length.

In some embodiments of the methods and recombinant genetic constructs according to the present disclosure, the inhibitor of PTBP1 comprises a PTBP1 guide RNA and Cas protein (or a nucleic acid molecule encoding the Cas protein).

The PTBP1 guide RNA and Cas protein may comprise a CRISPR interference system. As used herein, the term “CRISPR interference” or “CRISPRi” refers to a system that allows for sequence-specific repression of gene expression. CRISPRi systems comprise nuclease dead Cas (“dCas”) proteins (i.e., nuclease-inactivated Cas proteins) to block the transcription of a target gene, without cutting the target DNA sequence. Nuclease inactivated Cas proteins and methods of generating nuclease-inactivated Cas proteins are well known in the art (see, e.g., Qi et al., “Repurposing CRISPR as an RNA-Guided Platform for Sequence-Specific Control of Gene Expression,” Cell 152 (5): 1173-1183 (2013), which is hereby incorporated by reference in its entirety). Suitable nuclease dead Cas (dCas) proteins include, e.g., dCas9, dCas12a, and dCas12b

In some embodiments, the nuclease dead Cas (dCas) protein is a fusion protein comprising a Cas protein and one or more epigenetic modulators that suppress or silence the expression of the target gene, e.g., PTBP1. Suitable epigenetic modulators include, without limitation, DNA methyltransferase enzymes (e.g., DNA methyltransferase 3 alpha (“DNMT3A”) and DNA methyltransferase 3 like (“DNMT3L”)), histone demethylation enzymes (e.g., lysine-specific histone demethylase 1 (“LSD1”)), histone methyltransferase enzymes (e.g., G9A and SuV39h1), transcription factor recruitment domains (e.g., Krüppel-associated box domain (“KRAB”), KRAB-Methyl-CpG binding protein 2 domain (“KRAB-MeCP2”), enhancer of Zeste 2 (“EZH2”)), zinc finger transcriptional repressor domains (e.g., spalt like transcription factor 1 (“SALL1”) and suppressor of defective silencing protein 3 (“SDS3”), G9A, and Suv39h1) (see, e.g., Brezgin et al., “Dead Cas Systems: Types, Principles, and Applications,” Int. J. Mol. Sci. 20:6041 (2019); Yeo et al., “An Enhanced CRISPR Repressor for Targeted Mammalian Gene Regulation,” 15 (8): 611-616 (2018); Alerasool et al., “An Efficient KRAB Domain for CRISPRi Applications in Human Cells,” Nature Methods 17:1093-1096 (2020); and Duke et al., “An Improved CRISPR/dCas9 Interference Tool for Neuronal Gene Suppression,” Frontiers in Genome Editing 2:9 (2020), which are hereby incorporated by reference in their entirety).

In some embodiments, the epigenetic modulator is selected from the group consisting of Tet methylcytosine dioxygenase 1 (“TET1”), SunTag-TET1, MS2/MCP-TET1, p300Core, four tandem copies of herpes simplex viral protein 16 (“VP64”), VP160, NF-KB p65 activation domain (“p65”), Epstein-Barr Virus-derived R transactivator (“Rta”), SunTag-VP64, VP64-p65-Rta (“VPR”), SunTag-p65-HSF1, TV, synergistic activation mediator (“SAM”), Three-Component Repurposed Technology for Enhanced Expression (“TREE”), Casilio, Scaffold, and CMV (see, e.g., Brezgin et al., “Dead Cas Systems: Types, Principles, and Applications,” Int. J. Mol. Sci. 20(23): 6041 (2019), which is hereby incorporated by reference in its entirety). In some embodiments, when demethylation of a gene or gene protein is effective to suppress its transcription, the epigenetic modulator is a demethylase (e.g., TET1).

In any embodiment, the nuclease dead Cas protein is fused to a methyltransferase. In any embodiment, the nuclease dead Cas fusion protein is fused to a demethylase.

Suitable nuclease dead Cas fusion proteins are identified in Table 2 below.

TABLE 2
Exemplary Cas Fusion Proteins

| Fusion Protein | Reference* |
|---|---|
| dCas9-DNMT3A | Vojta et al., “Repurposing the CRISPR-Cas9 System for Targeted DNA |
| | Methylation,” Nucleic Acids Res. 44(12): 5615-5628 (2016) |
| dCas-SunTag- | Huang et al., “DNA Epigenome Editing using CRISPR-Cas SunTag- |
| DNMT3A | Directed DNMT3A,” Genome Biol. 18(1): 176 (2017) |
| dCas9-EZH2 | O'Geen et al., “Ezh2-dCas9 and KRAB-dCas9 Enable Engineering of |
| | Epigenetic Memory in a Context-Dependent Manner,” Epigenet. |
| | Chromatin. 12(1): 26 (2019) |
| dCas9-DNMT3A-3L | Brezgin et al., “Dead Cas Systems: Types, Principles, and Applications,” |
| | Int. J. Mol. Sci. 20(23): 6041 (2019) |
| dCas9-KRAB | O'Geen et al., “Ezh2-dCas9 and KRAB-dCas9 Enable Engineering of |
| | Epigenetic Memory in a Context-Dependent Manner,” Epigenet. |
| | Chromatin. 12(1): 26 (2019) |
| dCas9-KRAB-MeCP2 | Yeo et al., “An Enhanced CRISPR Repressor for Targeted Mammalian |
| | Gene Regulation,” Nat. Methods. 15(8): 611-616 (2018) |
| dCas12a-KRAB | Liu et al., “Engineering Cell Signaling Using Tunable CRISPR-Cpf1- |
| | based Transcription Factors,” Nat. Commun. 8: 2095 (2017) |
| dCas9-Chromo | Gilbert et al., “CRISPR-Mediated Modular RNA-Guided Regulation of |
| Shadow domain of | Transcription in Eukaryotes,” Cell 154: 442-451 (2013) |
| HP1α (“dCas9-Cs”) | |
| dCas9-WRPW domain | Gilbert et al., “CRISPR-Mediated Modular RNA-Guided Regulation of |
| of Hes1 (“dCas9- | Transcription in Eukaryotes,” Cell 154: 442-451 (2013) |
| WRPW”) | |
| dCas9-TET1 | Liu et al., “Editing DNA Methylation in the Mammalian Genome,” Cell |
| | 167: 233-247 (2016) |
| dCas9-SunTag-TET1 | Morita et al., “Targeted DNA Demethylation in vivo using dCas9- |
| | Peptide Repeat and scFv-TET1 Catalytic Domain Fusions,” Nat. |
| | Biotechnol. 34: 1060-1065 (2016) |
| dCas9-MS2/MCP- | Xu et al., “A CRISPR-Based Approach for Targeted DNA |
| TET1 | Demethylation,” Cell Discov. 2: 16009 (2016) |
| dCas9-P300 Core | Hilton et al., “Epigenome Editing by a CRISPR/Cas9-Based |
| | Acetyltransferase Activates Genes from Promoter and Enhancers,” Nat. |
| | Biotechnol. 33(5): 510-517 (2015) |
| dCas9-VP64 | Gilbert et al., “CRISPR-Mediated Modular RNA-Guided Regulation of |
| | Transcription in Eukaryotes,” Cell 154: 442-451 (2013) |
| dCas9-VP160 | Cheng et al., “Multiplexed Activators of Endogenous Genes by CRISPR- |
| | on, an RNA-Guided Transcriptional Activator System,” Cell Res. 23: |
| | 1163 (2013) |
| dCas9-p65 | Gilbert et al., “CRISPR-Mediated Modular RNA-Guided Regulation of |
| | Transcription in Eukaryotes,” Cell 154: 442-451 (2013) |
| dCas9-VPR | Chavez et al., “Highly Efficient Cas9-Mediated Transcriptional |
| | Programming,” Nat. Methods. 12: 326 (2015) |
| dCas9 SAM system | Konermann et al., “Genome-Scale Transcriptional Activation by an |
| | Engineered CRISPR-Cas9 Complex,” Nature 517(7536): 583-588 |
| | (2015) |
| dCas9 TREE system | Kunii et al., “Three-Component Repurposed Technology for Enhanced |
| | Expression: Highly Accumulable Transcriptional Activators via |
| | Branched Tag Arrays,” CRISPR J. 1(5): 337-347 (2018) |
| dCas9-TV (i.e., | Li et al., “A potent Cas9-Derived Gene Activator for Plant and |
| dCas9-6TAL-VP128 | Mammalian Cells,” Nat. Plants. 3: 930-936 (2017) |
| dCas12a-VPR | Liu et al., “Engineering Cell Signaling Using Tunable CRISPR-Cpf1- |
| | Based Transcription Factors,” Nat. Commun. 8: 2095 (2017) |
| dCas12a-p65 | Tak et al., “Inducible and Multiplex Gene Regulation Using CRISPR- |
| | Cpf1-Based Transcription Factors,” Nat. Methods 14: 1163-1166 (2017) |

*Each of which is hereby incorporated by reference in its entirety.

In some embodiments, the inhibitor of PTBP1 is a ZFN. A ZFN is an artificial endonuclease that comprises at least 1 zinc finger motif (e.g., at least 2, 3, 4, or 5 zinc finger motifs) fused to a nuclease domain (e.g., the cleavage domain of the FokI restriction enzyme). Heterodimerization of two individual ZFNs at a target nucleic acid sequence can result in cleavage of the target sequence. For example, two individual ZFNs may bind opposite strands of a target DNA sequence to induce a double-strand break in the target nucleic acid sequence. Methods of designing suitable ZFNs for inclusion in the systems of the presently claimed disclosure are well known in the art (see, e.g., Urnov et al., “Genome Editing with Engineered Zinc Finger Nucleases,” Nat. Rev. Genet. 11(9): 636-646 (2010); Gaj et al., “Targeted Gene Knockout by Direct Delivery of Zinc-Finger Nuclease Proteins,” Nat. Methods 9(8): 805-807 (2012); U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; and 6,479,626, which are hereby incorporated by reference in their entirety). In some embodiments, the first and second gene editing nucleases are FokI nucleases. In accordance with such embodiments, the first and second DNA binding motifs are zinc finger motifs.

In some embodiments, the inhibitor of PTBP1 is a TALEN. A TALEN is an engineered transcription activator-like effector nuclease that comprise a DNA-binding domain and a nuclease domain (e.g., a cleavage domain of the FokI restriction enzyme). The DNA-binding domain comprises a series of 33-35 amino acid repeat domains that each recognize a single base pair. Heterodimerization of two individual TALENs at a target nucleic acid sequence can result in cleavage of the target sequence. For example, two individual TALENs may bind opposite strands of a target DNA sequence to induce a double-strand break in the target nucleic acid sequence. Methods of designing suitable TALENs for inclusion in the systems of the presently claimed disclosure are well known in the art (see, e.g., Scharenberg et al., “Genome Engineering with TAL-Effector Nucleases and Alternative Modular Nuclease Technologies,” Curr. Gene Ther. 13(4): 291-303 (2013); Gaj et al., “Targeted Gene Knockout by Direct Delivery of Zinc-Finger Nuclease Proteins,” Nat. Methods 9(8): 805-807 (2012); Beurdeley et al., “Compact Designer TALENs for Efficient Genome Engineering,” Nat. Commun. 4:1762 (2013); U.S. Pat. Nos. 8,440,431; 8,440,432; 8,450,471; 8,586,363; and 8,697,853, which are hereby incorporated by reference in their entirety). In some embodiments, the first and second gene editing nucleases are FokI nucleases. In accordance with such embodiments, the first and second DNA binding motifs are TALE motifs.

The transcriptional repressor element-1 (RE1) silencing transcription factor (REST)/neuron-restrictive silencer factor (NRSF) is a gene silencing transcription factor that is widely expressed during embryogenesis and is critical to elaboration of the neuronal phenotype (Noh et al., “Repressor Element-1 Silencing Transcription Factor (REST)-Dependent Epigenetic Remodeling is Critical to Ischemia-Induced Neuronal Death,” PNAS 16: E962-E971 (2012), which is hereby incorporated by reference in its entirety). REST binds Neuron Restrictive Silencer Elements (NRSEs) in >2000 neuronal genes and represses their expression (Conaco et al., “Reciprocal Actions of REST and a microRNA Promote Neuronal Identity,” PNAS 103 (7): 2422-2427 (2006) and Schoenherr & Anderson, “The Neuron-Restrictive Silencer Factor (NRSF): A Coordinate Repressor of Multiple Neuron-Specific Genes,” Science 267:1360-1363 (1995), which are hereby incorporated by reference in their entirety). In some embodiments of the methods and recombinant genetic constructs according to the present disclosure, the one or more neuronal reprogramming factors is an inhibitor of RE1-silencing transcription factor (REST).

Suitable inhibitors of REST include, without limitation, (i) a REST siRNA or REST shRNA and (ii) a REST guide RNA and Cas protein.

In some embodiments of the methods and recombinant genetic constructs according to the present disclosure, the REST siRNA or REST shRNA is designed to target the sequence of Homo sapiens REI silencing transcription factor (REST), transcript variant 2, mRNA (NCBI Reference Sequence: NM_001193508.1, which is hereby incorporated by reference in its entirety), or a portion thereof. In some embodiments of the methods and recombinant genetic constructs according to the present disclosure, the REST siRNA or REST shRNA is designed to target the sequence of Homo sapiens RE1 silencing transcription factor (REST), transcript variant 3, mRNA (NCBI Reference Sequence: NM_001363453.2, which is hereby incorporated by reference in its entirety), or a portion thereof. In some embodiments of the methods and recombinant genetic constructs according to the present disclosure, the REST siRNA or REST shRNA is designed to target the sequence of Homo sapiens REI silencing transcription factor (REST), transcript variant 1, mRNA (NCBI Reference Sequence: NM_005612.5, which is hereby incorporated by reference in its entirety), or a portion thereof.

In some embodiments of the methods and recombinant genetic constructs according to the present disclosure, the inhibitor of REST comprises a REST guide RNA and Cas protein (or a nucleic acid molecule encoding the Cas protein). In accordance with such embodiments, the guide RNA comprises a nucleotide sequence that is complementary to a portion of the nucleic acid sequence of NCBI Reference Sequence Nos. NM_001193508.1, NM_001363453.2, or NM_005612.5, which are hereby incorporated by reference in their entirety. Suitable Cas proteins and nucleic acid molecules encoding said Cas proteins are described in detail supra.

In some embodiments of the methods and recombinant genetic constructs according to the present disclosure, the one or more neuronal reprogramming factors comprise one or more transcription factors. The term “transcription factor” refers to a DNA-binding protein that regulate the expression of specific genes. In some embodiments, the transcription factor is selected from the group consisting of CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, LHX6, ASCL1, SOX2, NEUROD1, NEUROD2, ISL1, LHX3, FOXG1, DLX5, NKX2.2, FEV, GATA2, LMX1B, FOXA2, and NGN2.

In some embodiments of the methods and recombinant genetic constructs according to the present disclosure, the transcription factor is ASCL1 having the sequence of SEQ ID NO: 6 below.

(SEQ ID NO: 6)
ATGGAAAGCTCTGCCAAGATGGAGAGCGGCGGCGCCGGCCAGCAGCCCCAGCCGCAGCCCCAGC
AGCCCTTCCTGCCGCCCGCAGCCTGTTTCTTTGCCACGGCCGCAGCCGCGGCGGCCGCAGCCGC
CGCAGCGGCAGCGCAGAGCGCGCAGCAGCAGCAGCAGCAGCAGCAGCAGCAGCAGCAGGCGCCG
CAGCTGAGACCGGCGGCCGACGGCCAGCCCTCAGGGGGCGGTCACAAGTCAGCGCCCAAGCAAG
TCAAGCGACAGCGCTCGTCTTCGCCCGAACTGATGCGCTGCAAACGCCGGCTCAACTTCAGCGG
CTTTGGCTACAGCCTGCCGCAGCAGCAGCCGGCCGCCGTGGCGCGCCGCAACGAGCGCGAGCGC
AACCGCGTCAAGTTGGTCAACCTGGGCTTTGCCACCCTTCGGGAGCACGTCCCCAACGGCGCGG
CCAACAAGAAGATGAGTAAGGTGGAGACACTGCGCTCGGCGGTCGAGTACATCCGCGCGCTGCA
GCAGCTGCTGGACGAGCATGACGCGGTGAGCGCCGCCTTCCAGGCAGGCGTCCTGTCGCCCACC
ATCTCCCCCAACTACTCCAACGACTTGAACTCCATGGCCGGCTCGCCGGTCTCATCCTACTCGT
CGGACGAGGGCTCTTACGACCCGCTCAGCCCCGAGGAGCAGGAGCTTCTCGACTTCACCAACTG
GTTC

In some embodiments of the methods and recombinant genetic constructs according to the present disclosure, the transcription factor is BRN2 having the sequence of SEQ ID NO: 7 below.

(SEQ ID NOL: 7)
ATGGCGACCGCAGCGTCTAACCACTACAGCCTGCTCACCTCCAGCGCCTCCATCGTACATGCCG
AGCCGCCTGGCGGCATGCAGCAGGGCGCAGGGGGCTACCGCGAGGCGCAGAGCCTGGTGCAGGG
CGACTACGGCGCGCTGCAGAGCAACGGGCACCCGCTCAGCCACGCTCACCAGTGGATCACCGCG
CTGTCCCACGGCGGCGGCGGCGGGGGCGGCGGCGGCGGTGGAGGAGGCGGGGGAGGCGGCGGGG
GAGGCGGCGACGGCTCCCCGTGGTCCACCAGCCCCCTAGGCCAGCCGGACATCAAGCCCTCGGT
GGTGGTACAGCAGGGTGGCCGAGGCGACGAGCTGCACGGGCCAGGAGCGCTGCAGCAACAGCAT
CAACAGCAACAGCAACAGCAGCAGCAGCAGCAGCAGCAGCAGCAGCAGCAACAGCAGCAGCAAC
AACAGCGACCGCCACATCTGGTGCACCACGCTGCCAACCACCATCCCGGGCCCGGGGCATGGCG
GAGTGCGGCGGCTGCAGCTCACCTCCCTCCCTCCATGGGAGCTTCCAACGGCGGTTTGCTCTAT
TCGCAGCCGAGCTTCACGGTGAACGGCATGCTGGGCGCAGGAGGGCAGCCGGCTGGGCTGCACC
ACCACGGCCTGAGGGACGCCCACGATGAGCCACACCATGCAGACCACCACCCGCATCCGCACTC
TCACCCACACCAGCAACCGCCCCCGCCACCTCCCCCACAAGGCCCACCGGGCCACCCAGGCGCG
CACCACGACCCGCACTCGGACGAGGACACGCCGACCTCAGACGACCTGGAGCAGTTCGCCAAGC
AATTCAAGCAGAGGCGGATCAAACTCGGATTTACTCAAGCAGACGTGGGGCTGGCGCTTGGCAC
CCTGTACGGCAACGTGTTCTCGCAGACCACCATCTGCAGGTTTGAGGCCCTGCAGCTGAGCTTC
AAGAACATGTGCAAGCTGAAGCCTTTGTTGAACAAGTGGTTGGAAGAGGCAGACTCATCCTCGG
GCAGCCCCACCAGCATAGACAAGATCGCAGCGCAAGGGCGCAAACGGAAAAAGCGGACCTCCAT
CGAGGTGAGCGTCAAGGGGGCTCTGGAGAGCCATTTCCTCAAATGCCCTAAGCCCTCGGCCCAG
GAGATCACCTCCCTCGCGGACAGCTTACAGCTGGAGAAGGAGGTGGTGAGAGTTTGGTTTTGTA
ACAGGAGACAGAAAGAGAAAAGGATGACCCCTCCCGGAGGGACTCTGCCGGGCGCCGAGGATGT
GTATGGGGGTAGTAGGGACACGCCACCACACCACGGGGTGCAGACGCCCGTCCAGT

In some embodiments of the methods and recombinant genetic constructs according to the present disclosure, the transcription factor is LMX1A having the sequence of SEQ ID NO: 9 below.

(SEQ ID NO: 9)
ATGCTGGACGGCCTAAAGATGGAGGAGAACTTCCAAAGCGCGATCGACACCTCGGCCTCCTTCT
CCTCGCTGCTGGGCAGAGCGGTGAGCCCCAAGTCTGTCTGCGAGGGCTGTCAGCGGGTCATCTT
GGACAGGTTTCTGCTGCGGCTCAACGACAGCTTCTGGCATGAGCAGTGCGTGCAGTGCGCCTCC
TGCAAAGAGCCCCTGGAGACCACCTGCTTCTACCGGGACAAGAAGCTGTACTGCAAGTATGACT
ACGAGAAGCTGTTTGCTGTTAAATGTGGGGGCTGCTTCGAGGCCATCGCTCCCAATGAGTTTGT
TATGCGGGCCCAGAAGAGTGTATACCACCTGAGCTGCTTCTGTTGCTGTGTCTGCGAGCGACAG
CTTCAGAAGGGTGATGAGTTTGTCCTGAAGGAGGGGCAGCTGCTCTGCAAAGGGGACTATGAGA
AGGAGCGGGAGCTACTCAGCCTGGTGAGCCCAGCAGCCTCAGACTCAGGTAAAAGTGATGATGA
AGAAAGTCTCTGCAAGTCAGCCCATGGGGCAGGGAAAGGAACTGCTGAGGAAGGCAAGGACCAT
AAGCGCCCCAAACGTCCGAGAACCATCTTGACAACTCAACAGAGGCGAGCATTCAAGGCCTCAT
TTGAAGTATCCTCCAAGCCCTGCAGGAAGGTGAGAGAGACTCTGGCTGCAGAGACAGGGCTGAG
TGTCCGTGTCGTCCAGGTGTGGTTCCAAAACCAGAGAGCGAAGATGAAGAAGCTGGCCAGGCGA
CAGCAGCAGCAGCAGCAAGATCAGCAGAACACCCAGAGGCTGAGCTCTGCTCAGACAAACGGTG
GTGGGAGTGCTGGGATGGAAGGAATCATGAACCCCTACACGGCTCTGCCCACCCCACAGCAGCT
CCTGGCCATCGAGCAGAGTGTCTACAGCTCAGATCCCTTCCGACAGGGTCTCACCCCACCCCAG
ATGCCTGGAGACCACATGCACCCTTATGGTGCCGAGCCCCTTTTCCATGACCTGGATAGCGACG
ACACCTCCCTCAGTAACCTGGGTGACTGTTTCCTAGCAACCTCAGAAGCTGGGCCTCTGCAGTC
CAGAGTGGGAAACCCCATTGACCATCTGTACTCCATGCAGAATTCTTACTTCACATCT

In some embodiments of the methods and recombinant genetic constructs according to the present disclosure, the transcription factor is LHX6 having the sequence of SEQ ID NO: 10 below.

(SEQ ID NO: 10)
ATGTACTGGAAGCATGAGAACGCCGCCCCGGCGTTGCCCGAGGGCTGCCCCGCCACCGACCAGG
TGATGGCCCAGCCAGGGTCCGGCTGCAAAGCGACCACCCGCTGTCTTGAAGGGACCGCGCCGCC
CGCCATGGCTCAGTCTGACGCCGAGGCCCTGGCAGGAGCTCTGGACAAGGACGAGGGTCAGGCC
TCCCCATGTACGCCCAGCACGCCATCTGTCTGCTCACCGCCCTCTGCCGCCTCCTCCGTGCCGT
CTGCAGGCAAGAACATCTGCTCCAGCTGCGGCCTCGAGATCCTGGACCGATATCTGCTCAAGGT
CAACAACCTCATCTGGCACGTGCGGTGCCTCGAGTGCTCCGTGTGTCGCACGTCGCTGAGGCAG
CAGAACAGCTGCTACATCAAGAACAAGGAGATCTTCTGCAAGATGGACTACTTCAGCCGATTCG
GGACCAAGTGTGCCCGGTGCGGCCGACAGATCTACGCCAGCGACTGGGTGCGGAGAGCTCGCGG
CAACGCCTACCACCTGGCCTGCTTCGCCTGCTTCTCGTGCAAGCGCCAGCTGTCCACTGGTGAG
GAGTTCGGCCTGGTCGAGGAGAAGGTGCTCTGCCGCATCCACTACGACACCATGATTGAGAACC
TCAAGAGGGCCGCCGAGAACGGGAACGGCCTCACGTTGGAGGGGGCAGTGCCCTCGGAACAGGA
CAGTCAACCCAAGCCGGCCAAGCGCGCGCGGACGTCCTTCACCGCGGAACAGCTGCAGGTTATG
CAGGCGCAGTTCGCGCAGGACAACAACCCCGACGCTCAGACGCTGCAGAAGCTGGCGGACATGA
CGGGCCTCAGCCGGAGAGTCATCCAGGTGTGGTTTCAAAACTGCCGGGCGCGTCATAAAAAGCA
CACGCCGCAACACCCAGTGCCGCCCTCGGGGGCGCCCCCGTCCCGCCTTCCCTCCGCCCTGTCC
GACGACATCCACTACACCCCGTTCAGCAGCCCCGAGCGGGCGCGCATGGTCACCCTGCACGGCT
ACATTGAGAGTCAGGTACAGTGCGGGCAGGTGCACTGCCGGCTGCCTTACACCGCACCCCCCGT
CCACCTCAAAGCCGATATGGATGGGCCGCTCTCCAACCGGGGTGAGAAGGTCATCCTTTTTCAG
TAC

Nucleic acid sequences and molecules encoding the one or more transcription factors identified herein are well known and accessible in the art.

Exemplary nucleic acid sequences encoding a transcription factor of the present disclosure and amino acid sequences of the transcription factors of the present disclosure are set forth in Table 3 below.

TABLE 3
Exemplary Transcription Factor Sequences

| Transcription Factor | NCBI Reference. Sequence* |
|---|---|
| CTIP (Homo sapiens BAF chromatin remodeling complex | NM_001282237.2 |
| subunit BCL11B (BCL11B), transcript variant 3, mRNA) | NP_001269166.1 |
| Homo sapiens distal-less homeobox 1 (DLX1), transcript | NM_178120.5 |
| variant 1, mRNA | NP_835221.2 |
| Homo sapiens distal-less homeobox 2 (DLX2), mRNA | NM_004405.4 |
| | NP_004396.1 |
| Homo sapiens myelin transcription factor 1 like (MYT1L), | NM_001303052.2 |
| transcript variant 1, mRNA | NP_001289981.1 |
| Homo sapiens forkhead box P1 (FOXP1), transcript variant | NM_001349338.3 |
| 11, mRNA | NP_001336267.1 |
| Homo sapiens forkhead box P2 (FOXP2), transcript variant | NM_014491.4 |
| 1, mRNA | NP_055306.1 |
| Mus musculus zinc finger protein 503 (Zfp503), mRNA | NM_145459.3 |
| | NP_663434.2 |
| Homo sapiens retinoic acid receptor beta (RARB), | NM_000965.5 |
| transcript variant 1, mRNA | NP_000956.2 |
| Homo sapiens retinoid X receptor gamma (RXRG), | NM_006917.5 |
| transcript variant 1, mRNA | NP_008848.1 |
| Homo sapiens homeobox protein GSH-2 (GSH2) gene, | AF439445.1 |
| complete cds | AAM08285.1 |
| Homo sapiens achaete-scute family bHLH transcription | NM_004316.4 |
| factor 1 (ASCL1), mRNA | NP_004307.2 |
| BRN2 (Homo sapiens) POU class 3 homeobox 2 | NM_005604.4 |
| (POU3F2), mRNA | NP_005595.2 |
| Homo sapiens Zic family member 1 (ZIC1), mRNA | NM_003412.4 |
| | NP_003403.2 |
| Homo sapiens oligodendrocyte transcription factor 2 | NM_005806.4 |
| (OLIG2), mRNA | NP_005797.1 |
| NGN2 (Homo sapiens neurogenin 2 (NEUROG2), mRNA) | NM_024019.4 |
| | NP_076924.1 |
| NURR1 (Homo sapiens Nurr1 gene, complete cds) | AB017586.1 |
| | BAA75666.1 |
| Homo sapiens LIM homeobox transcription factor 1 alpha | NM_177398.4 |
| (LMX1A), transcript variant 1, mRNA | NP_796372.1 |
| Homo sapiens SRY-box transcription factor 2 (SOX2), | NM_003106.4 |
| mRNA | NP_003097.1 |
| Homo sapiens neuronal differentiation 1 (NEUROD1), | NM_002500.5 |
| transcript variant 1, mRNA | NP_002491.3 |
| Homo sapiens neuronal differentiation 2 (NEUROD2), | NM_006160.4 |
| mRNA | NP_006151.3 |
| Homo sapiens ISL LIM homeobox 1 (ISL1), mRNA | NM_002202.3 |
| | NP_002193.2 |
| Homo sapiens LIM homeobox 3 (LHX3), transcript variant | NM_178138.6 |
| 1, mRNA | NP_835258.1 |
| Homo sapiens forkhead box G1 (FOXG1), mRNA | NM_005249.5 |
| | NP_005240.3 |
| Homo sapiens distal-less homeobox 5 (DLX5), mRNA | NM_005221.6 |
| | NP_005212.1 |
| Homo sapiens NK2 homeobox 2 (NKX2-2), mRNA | NM_002509.4 |
| | NP_002500.1 |
| Homo sapiens FEV transcription factor, ETS family | NM_017521.3 |
| member (FEV), mRNA | NP_059991.1 |
| Homo sapiens GATA binding protein 2 (GATA2), | NM_032638.5 |
| transcript variant 2, mRNA | NP_116027.2 |
| Homo sapiens LIM homeobox transcription factor 1 beta | NM_001174147.2 |
| (LMX1B), transcript variant 2, mRNA | NP_001167618.1 |
| Homo sapiens forkhead box A2 (FOXA2), transcript | NM_021784.5 |
| variant 1, mRNA | NP_068556.2 |
| Homo sapiens neurogenin 2 (NEUROG2), mRNA | NM_024019.4 |
| | NP_076924.1 |

*Each of which is hereby incorporated by reference in its entirety.

As described supra, neurodegenerative disorders comprise a heterogeneous category, that include both multicentric and diffuse disorders such as Alzheimer's, and those in which the loss of a single phenotype predominates, such as Huntington's and Parkinson's diseases (Goldman, S. A., “Stem and Progenitor Cell-Based Therapy of the Central Nervous System: Hopes, Hype and Wishful Thinking,” Cell Stem Cell 18(2): 174-188 (2016), which is hereby incorporated by reference in its entirety).

Without being bound by theory, expression of one or more of the neuronal reprogramming factors disclosed herein in a population of glial progenitor cells may be effective to generate a population of neurons (e.g., striatal medium spiny neurons or dopaminergic nigrostriatal neurons) suitable for the treatment of neurodegenerative disorders including, without limitation, Huntington's and Parkinson's diseases. Accordingly, the recombinant genetic construct according to the present disclosure may comprise a nucleic acid sequence encoding one or more neuronal reprogramming factors selected from the group consisting of a microRNA, an inhibitor of PTBP1, an inhibitor of REST, and one or more transcription factors.

In any embodiment of the methods and recombinant genetic construct disclosed herein, the one or more transcription factors may comprise CTIP2, DLX1, DLX2, MYT1L, or a combination thereof. In accordance with such embodiments, the one or more neuronal reprogramming factors comprise miR-9/9*, miR-124, CTIP2, DLX1, DLX2, MYT1L, or a combination thereof. Expression of miR-9/9* and miR-124 (miR-9/9*-124) together with CTIP2 (also known as BCL11B), DLX1, DLX2, and MYT1L has been shown to guide the conversion of human postnatal and adult fibroblasts into an enriched population of neurons analogous to striatal medium spiny neurons (MSNs) (Victor et al., “Generation of Human Striatal Neurons by microRNA-Dependent Direct Conversion of Fibroblasts,” Neuron 84(2): 311-323 (2014), which is hereby incorporated by reference in its entirety).

In any embodiment of the methods and recombinant genetic construct disclosed herein, the one or more transcription factors comprise ASCL1, BRN2, MYTL1, or a combination thereof. The combination of ASCL1, BRN2A, and MYT1 has been shown to reprogram mouse fibroblasts to functional neurons (Grealish et al., “Brain Repair and Reprogramming: The Route to Clinical Translation,” J. Internal Med 280:265-275 (2016), which is hereby incorporated by reference in its entirety).

In any embodiment of the methods and recombinant genetic construct disclosed herein, the one or more transcription factors comprise ASCL1, NURR1, LMX1A, or a combination thereof. In accordance with such embodiments, the one or more neuronal reprogramming factors comprise a REST inhibitor, ASCL1, NURR1, LMX1A, or a combination thereof. The use of a short hairpin RNA against the REI-silencing transcription factor (REST) complex, together with ACL1, LMX1A, and NURR1 (together referred to as ALN) has recently been shown to reprogram human glial progenitor cells into induced dopaminergic neurons, which at three weeks following transduction, expressed DA-related genes, including TH, SLC6A3 (DAT), FOXA2, LMX1A, and PITX3 (Nolbrant et al., “Direct Reprogramming of Human Fetal- and Stem Cell-Derived Glial Progenitor Cells into Midbrain Dopaminergic Neurons,” Stem Cell Reports 15(4): 869-882 (2020), which is hereby incorporated by reference in its entirety). Addition of FOXA2 resulted in a higher endogenous expression of the midbrain dopaminergic genes LMX1A, EN1, and OTX2 (Nolbrant et al., “Direct Reprogramming of Human Fetal- and Stem Cell-Derived Glial Progenitor Cells into Midbrain Dopaminergic Neurons,” Stem Cell Reports 15(4): 869-882 (2020), which is hereby incorporated by reference in its entirety). Thus, in some embodiments, the one or more neuronal reprogramming factors comprise a REST inhibitor, ASCL1, NURR1, LMX1A, FOXA2, or a combination thereof.

In any embodiment of the methods and recombinant genetic construct disclosed herein, the one or more neuronal reprogramming factors comprise the transcription factors ISL1 and/or LHX3. In accordance with such embodiments, the one or more neuronal reprogramming factors comprise miR-9/9*, miR-124, ISL1, LHX3, or a combination thereof (e.g., miR-9/9*, miR-124, and ISL1 or miR-9/9*, miR-124, and LHX3). The use of the motor neuron transcription factors ISL1 and LHX3 in combination with miR-9/9* and miR-124 has been shown to mediate the conversion of human fibroblasts to motor neurons (see, e.g., U.S. Patent Application Publication No. 2002/0377885 to Yoo et al. and Lu & Yoo, “Mechanistic Insights Into MicroRNA-Induced Neuronal Reprogramming of Human Adult Fibroblasts,” Front. Neurosci. 12:522 (2018), which are hereby incorporated by reference in their entirety).

In any embodiment of the methods and recombinant genetic construct disclosed herein, the one or more neuronal reprogramming factors comprise the transcription factors SOX2 alone or in combination with ASCL1. Expression of the transcription factor SOX2 alone or in combination with the transcription factor ASCL1, has been shown to induce the conversion of genetically fate-mapped NG2 glia into induced doublecortin” neurons in the adult mouse cerebral cortex (Heinrich et al., “Sox2-Mediated Conversion of NG2 Glia into Induced Neurons in the Injured Adult Cerebral Cortex,” Stem Cell Reports 3(6): 10001014 (2014), which is hereby incorporated by reference in its entirety).

In any embodiment of the methods and recombinant genetic construct disclosed herein, the one or more neuronal reprogramming factors comprise the transcription factor NEUROD1. Studies have shown that in vivo retroviral expression of NEUROD1 mediates the direct reprogramming of NG2 cells into glutamatergic and GABAergeic neurons (Guo et al., “In Vivo Direct Reprogramming of Reactive Glial Cells into Functional Neurons After Brain Injury and in an Alzheimer's Disease Model,” Cell Stem Cell 14:188-202 (2014), which is hereby incorporated by reference in its entirety).

As described herein, regulation of transcription in prokaryotic cells typically involves operons. The term “operon” refers to a region of DNA that consists of one or more genes that encode the proteins needed for a specific function. The operon also includes a promoter and an operator. The term “operator” refers to a region of the operon where regulatory proteins bind. Typically, the operator is located near the promoter and helps regulate the transcription of the operon genes.

A well-known example of operon regulation involves the tet operon in E. coli. The tet operon consists of a promoter, an operator, and genes that encode proteins needed to confer resistance to the antibiotic tetracycline. The TetR repressor protein binds to an operator site and so prevents expression of the tetracycline resistance genes. The term “repressor” refers to any protein that binds to DNA and thus regulates the expression of genes by decreasing the rate of transcription. When tetracycline is present, it binds to the TetR protein instead of the operator site. Consequently, the tet operon is induced by tetracycline. The tet system and other well-known operator systems may be incorporated into any of the recombinant genetic constructs disclosed herein. Suitable operator systems are disclosed in more detail infra.

In any embodiment of the methods and recombinant genetic construct disclosed herein, the promoter and/or enhancer of the construct is operably linked to an inducible promoter and/or operator system sequence.

An inducible promoter and/operator system is capable of directly or indirectly activating transcription of the nucleic acid molecule that it is operably linked to in response to a “regulatory agent” (e.g., a chemical agent or biological molecule, such as a metabolite, a small molecule) or a stimulus. In the absence of a “regulatory agent” or stimulus, the first and/or second nucleic acid molecules of the system will not be transcribed. The term “not transcribed” or “not substantially expressed” means that the level of transcription is at least 50-fold lower than the level of transcription observed in the presence of an appropriate stimulus or regulatory agent; and preferably at least 10-fold, 250-fold, or 500-fold or lower than the level of transcription observed in the presence of an appropriate stimulus or regulatory agent.

Inducible promoters and/or operator systems that may be used in performing the methods or included in the systems of the present disclosure include those regulated by hormones and hormone analogs such as progesterone, ecdysone and glucocorticoids as well as promoters and/operators which are regulated by tetracycline, heat shock, heavy metal ions, interferon, and lactose operon activating compounds. For a review of these systems see Gingrich & Roder, “Inducible Gene Expression in the Nervous System of Transgenic Mice,” Annu. Rev. Neurosci. 21:377-405 (1998), which is hereby incorporated by reference in its entirety. Tissue-specific expression has been well characterized in the field of gene expression and tissue-specific and other inducible promoters are well known in the art.

In accordance with the systems and recombinant genetic constructs disclosed herein, the inducible promoter and/or operator systems according to the present disclosure control expression of the system, i.e., the dCas nuclease system or Cas nuclease system for producing neurons from glial progenitor cells. When reprogramming of the glial progenitor cells is desired, it is achieved by administering a suitable regulatory agent (e.g., doxycycline, tetracycline, hormone,) or other inducing agent to the subject, preferably using a route or other means that is targeted to the cells of the subject comprising the system. Contacting the glial progenitor cell comprising the recombinant genetic constructs described herein with a regulatory agent may induce expression of the one or more neuronal reprogramming factors for producing neurons from glial progenitor cells, e.g., miR-9/9*, miR-124, an inhibitor of polypyrimidine-tract-binding protein 1 (PTBP1), an inhibitor of RE1-silencing transcription factor (REST), and/or one or more transcription factors selected from the group consisting of CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, SOX2, NEUROD1, NEUROD2, ISL1, LHX3, FOXGI, DLX5, NKX2.2, FEV, GATA2, LMX1B, FOXA2, and NGN2. However, it should be recognized by one skilled in the art that in other inducible vectors, the opposite is true, that is, the regulatory agent inhibits expression and removal permits expression.

Suitable inducible promoter and/or operator systems for inclusion in the systems of the present disclosure are well known in the art and include, without limitation, a tetracycline-controlled operator system, a cumate-controlled operator system, a rapamycin inducible system, a FKCsA inducible system, and an ABA inducible system (see, e.g., Kallunki et al., “How to Choose the Right Inducible Gene Expression System for Mammalian Studies?” Cells 8 (8): 796 (2019); U.S. Pat. Nos. 8,728,759; and 7,745,592, which are hereby incorporated by reference in their entirety).

In some embodiments, the tetracycline-controlled operator system comprises a repression-based configuration, in which a Tet operator (“TetO”) is inserted between a constitutive promoter and gene of interest and where the binding of the Tet repressor (“TetR”) to the operator suppresses downstream transcription of a nucleic acid sequence of interest (see, e.g., Kallunki et al., “How to Choose the Right Inducible Gene Expression System for Mammalian Studies?” Cells 8 (8): 796 (2019), which is hereby incorporated by reference in its entirety). In accordance with such embodiments, the addition of tetracycline (or the synthetic tetracycline derivative doxycycline) results in the disruption of the association between TetR and TetO, thereby triggering TetO-dependent transcription of the nucleic acid sequence of interest.

In some embodiments, the tetracycline-controlled operator system comprises a Tet-off configuration, where tandem TetO sequences are positioned upstream of a minimal promoter followed by a nucleic acid sequence of interest (see, e.g., Kallunki et al., “How to Choose the Right Inducible Gene Expression System for Mammalian Studies?” Cells 8(8): 796 (2019), which is hereby incorporated by reference in its entirety). In accordance with such embodiments, a chimeric protein consisting of TetR and VP16 (“tTA”), a eukaryotic transactivator derived from herpes simplex virus type 1, is converted into a transcriptional activator, and the expression plasmid is transfected together with the operator plasmid. Thus, the presence of tetracycline (or the synthetic tetracycline derivative doxycycline) switches off the expression of the system or its components, while removing tetracycline switches it on.

In some embodiments, the tetracycline-controlled operator system comprises a Tet-on configuration, where the system or its components is transcribed when tetracycline is present (see, e.g., Kallunki et al., “How to Choose the Right Inducible Gene Expression System for Mammalian Studies?” Cells 8(8): 796 (2019), which is hereby incorporated by reference in its entirety). In accordance with such embodiments, tandem TetO sequences are positioned upstream of a minimal promoter followed by a nucleic acid sequence of interest. In the presence of tetracycline (or the synthetic tetracycline derivative doxycycline), a mutant rTa (“rtTa”) binds to TetO sequences, thereby activating the minimal promoter.

In some embodiments, the inducible promoter and/or operator system is a cumate-controlled operator system. Similar to the tetracycline-controlled operator system, the cumate-controlled operator system, the cumate operator (“CuO”) and its repressor (“CymR”) may be engineered into a repressor configuration, an activator configuration, and a reverse activator configuration (see, e.g., Kallunki et al., “How to Choose the Right Inducible Gene Expression System for Mammalian Studies?” Cells 8(8): 796 (2019), which is hereby incorporated by reference in its entirety).

In some embodiments, the cumate-controlled operator system comprises a repression-based configuration, in which the cumate repressor (“CymR”) is used to repress transcription from a mammalian promoter by binding an operator site (“CuO”) placed downstream of the initiation site (see Mullick et al., “The Cumate Gene-Switch: A System for Regulated Expression in Mammalian Cells,” BMC Biotechnol. 6:43 (2006), which is hereby incorporated by reference in its entirety). In accordance with such embodiments, addition of the inducer (cumate) to mammalian cells causes a change in the configuration of CymR such that it can no longer bind DNA and thus relieves repression, thereby triggering CuO-dependent expression of the system or system components.

In some embodiments, the cumate-controlled operator system comprises an activator configuration, in which CymR is fused to an activation domain and the chimeric molecule (“cTA”) is used to activate transcription from a minimal promoter downstream of multimerized operator binding sites (e.g., 6X-CuO) (see Mullick et al., “The Cumate Gene-Switch: A System for Regulated Expression in Mammalian Cells,” BMC Biotechnol. 6:43 (2006), which is hereby incorporated by reference in its entirety). cTa may be formed via the fusion of CymR and VP16. In accordance with such embodiments, binding of cTA, and therefore activation, is regulated by addition of cumate. Transcription of a nucleic acid sequence of interest is controlled by the minimal promoter, which is activated in the absence of cumate.

In some embodiments, the cumate-controlled operator system comprises a reverse activator configuration, where a nucleic acid sequence is transcribed when cumate is present. In accordance with such embodiments, tandem CuO sequences are positioned upstream of a minimal promoter followed by a nucleic acid sequence of interest. In the presence of cumate, a cTA mutant (“rcTA”) binds to CuO sequences, thereby activating the minimal promoter (see Mullick et al., “The Cumate Gene-Switch: A System for Regulated Expression in Mammalian Cells,” BMC Biotechnol. 6:43 (2006), which is hereby incorporated by reference in its entirety).

In some embodiments, the inducible promoter and/or operator system is a rapamycin inducible system. In accordance with such embodiments, the promoter is a rapamycin-inducible promoter (e.g., a minimal IL-2 promoter). In this system, a DNA binding domain (ZFHD1) and a transcription factor activation domain (NF-KB p65) are expressed separately as fusion proteins with the rapamycin-binding domains of FKBP12 and FRAP (mTOR), respectively (see, e.g., Koh et al., “Use of a Stringent Dimerizer-Regulated Gene Expression System for Controlled BMP2 Delivery,” Mol. Ther. 14(5): P684-691 (2006), which is hereby incorporated by reference in its entirety). On addition of rapamycin (or the rapamycin analog FK506), the fusion proteins are reversibly cross-linked to drive transcription of a nucleic acid sequence of interest. Mutation of the rapamycin-binding region of the mTOR-activation domain fusion protein results in systems responsive to rapamycin-like compounds (rapalogs) that, unlike rapamycin, do not bind to endogenous mTOR protein and, therefore, have little immunosuppressive or antiproliferative activity.

In any embodiment of the methods and recombinant genetic constructs disclosed herein, the construct is an expression vector. Suitable expression vectors include, without limitation, viral vectors. For example, the expression vector may be a viral vector selected from the group consisting of an adenovirus vector, an adeno-associated virus (“AAV”) vector, a retrovirus vector, a lentivirus vector, a vaccinia virus vector, a herpes virus vectors, and any other vector suitable for introduction of the encoded neuronal reprogramming factor(s) described herein into a given cell (e.g., a glial progenitor cell) by any means to facilitate expression of the encoded neuronal reprogramming factor(s). In some embodiments, the viral vector is selected from a lentiviral vector, an adeno-associated viral vector, vaccinia vector, and a retroviral vector.

In some embodiments, the vector is a lentiviral vector (see, e.g., Ura et al., “Developments in Viral Vector-Based Vaccines,” Vaccines 2:624-641 (2014) and Hu et al., “Immunization Delivered by Lentiviral Vectors for Cancer and Infection Diseases,” Immunol. Rev. 239:45-61 (2011), which are hereby incorporated by reference in their entirety).

In some embodiments, the vector is a retroviral vector (see e.g., Ura et al., “Developments in Viral Vector-Based Vaccines,” Vaccines 2:624-641 (2014), which are hereby incorporated by reference in their entirety), a vaccinia virus, a replication deficient adenovirus vector, and a gutless adenovirus vector (see e.g., U.S. Pat. No. 5,872,005, which is incorporated herein by reference in its entirety).

In other embodiments, the vector is an adeno-associated virus (AAV) vector (see, e.g., Krause et al., “Delivery of Antigens by Viral Vectors for Vaccination,” Ther. Deliv. 2(1): 51-70 (2011); Ura et al., “Developments in Viral Vector-Based Vaccines,” Vaccines 2:624-641 (2014); Buning et al, “Recent Developments in Adeno-associated Virus Vector Technology,” J. Gene Med. 10:717-733 (2008), each of which is incorporated herein by reference in its entirety).

Methods for generating and isolating viral expression vectors suitable for use as vectors are known in the art (see, e.g., Bulcha et al., “Viral Vector Platforms within the Gene Therapy Landscape,” Nature 6:53 (2021); Bouard et al., “Viral Vectors: From Virology to Transgene Expression,” Br. J. Pharmacol. 157(2): 153-165 (2009); Grieger & Samulski, “Adeno-associated Virus as a Gene Therapy Vector: Vector Development, Production and Clinical Applications,” Adv. Biochem. Engin Biotechnol. 99:119-145 (2005); Buning et al, “Recent Developments in Adeno-associated Virus Vector Technology,” J. Gene Med. 10:717-733 (2008), each of which is incorporated herein by reference in its entirety).

### Populations of Human Glial Progenitor Cells

Aspects of the present disclosure also relate to a population of human glial progenitor cells comprising a genetic construct according to the present disclosure.

As used herein, the term “glial cells” refers to a population of non-neuronal cells that provide support and nutrition, maintain homeostasis, either form myelin or promote myelination, and participate in signal transmission in the nervous system. The term “glial progenitor cells” refers to cells having the potential to differentiate into cells of the glial lineage such as oligodendrocytes and astrocytes (French-Constant and Raff, “Proliferating Bipotential Glial Progenitor Cells in Adult Rat Optic Nerve,” Nature 319:499-502 (1986) and Raff et al., “A Glial Progenitor Cell that Develops in Vitro into an Astrocyte or an Oligodendrocyte Depending on Culture Medium,” Nature 303:390-396 (1983), which are hereby incorporated by reference in their entirety).

The glial progenitor cells of the population may be astrocyte biased glial progenitor cells, oligodendrocyte-biased glial progenitor cells, unbiased glial progenitor cells, or a combination thereof. The glial progenitor cells of the population may express one or more markers of the glial cell lineage. For example, in one embodiment, the glial progenitor cells of the population may express A2B5⁺. In another embodiment, the glial progenitor cells of the population are positive for a PDGFαR marker. The PDGFαR marker is optionally a PDGFαR ectodomain, such as CD140a. PDGFαR and CD140a are markers of an oligodendrocyte-biased glial progenitor cells. In another embodiment, the glial progenitor cells of the population are CD44⁺. CD44 is a marker of an astrocyte-biased glial progenitor cell. In another embodiment, the glial progenitor cells of the population are positive for a CD9 marker. The CD9 marker is optionally a CD9 ectodomain. In one embodiment, the glial progenitor cells of the population are A2B5⁺, CD140a⁺, and/or CD44⁺. The aforementioned glial progenitor cell surface markers can be used to identify, separate, and/or enrich the population for glial progenitor cells prior to administration.

The glial progenitor cell population is optionally negative for a PSA-NCAM marker and/or other neuronal lineage markers, and/or negative for one or more inflammatory cell markers, e.g., negative for a CD11 marker, negative for a CD32 marker, and/or negative for a CD36 marker (which are markers for microglia). Optionally, the population of glial progenitor cells are negative for any combination or subset of these additional markers. Thus, for example, the population of glial progenitor cells is negative for any one, two, three, or four of these additional markers.

Glial progenitor cells of the population may be stably transduced with one or more of the recombinant genetic constructs described herein. In accordance with such embodiments, the glial progenitor cells of the population express at least one of the recombinant genetic constructs described herein. In some embodiments, the one or more neuronal reprogramming factors encoded by each of the one or more recombinant genetic constructs is not endogenously expressed by the glial progenitor cells of the population; however, the one or more neuronal reprogramming factors is expressed in a target cell-specific manner either via the activation of the promoter and/or enhancer for a gene which is selectively or specifically expressed by human glial progenitor cells according to the present disclosure.

The population of glial progenitor cells may be a population of glial progenitor cells from mammalian cells, e.g., a population of human cells.

Glial progenitor cells can be obtained from embryonic, fetal, or adult brain tissue, embryonic stem cells, or induced pluripotential cells. Suitable methods for obtaining glial progenitor cells from embryonic stem cells or induced pluripotent stem cells are known in the art, see e.g., U.S. Pat. No. 10,450,546 to Goldman and Wang, which is hereby incorporated by reference in its entirety.

Alternatively, the glial progenitor cells are isolated from ventricular and subventricular zones of the brain or from the subcortical white matter. Glial progenitor cells can be extracted from brain tissue containing a mixed population of cells directly by using the promoter specific separation technique, as described in U.S. Patent Application Publication Nos. 20040029269 and 20030223972 to Goldman, which are hereby incorporated by reference in their entirety. This method involves selecting a promoter which functions specifically in glial progenitor cells, and introducing a nucleic acid encoding a marker protein under the control of said promoter into the mixed population cells. The mixed population of cells is allowed to express the marker protein and the cells expressing the marker protein are separated from the population of cells, with the separated cells being the glial progenitor cells.

In some embodiments, the population of glial progenitor cells is a population of bi-potential glial progenitor cells.

In some embodiments, the population of glial progenitor cells is biased to producing oligodendrocytes. In accordance with such embodiments, the population of glial progenitor cells may be oligodendrocyte progenitor cells.

Alternatively, the glial progenitor cells are biased to producing astrocytes.

In any embodiment, cells of the population are transduced with a recombinant genetic construct comprising a promoter and/or enhancer for a gene which is selectively or specifically expressed by human glial progenitor cells (e.g., a platelet derived growth factor alpha (PDGFRA) promoter, a zinc finger protein 488 (ZNF488) promoter, a G protein-coupled receptor (GPR17) promoter, an oligodendrocyte Transcription Factor 2 (OLIG2) promoter, a chondroitin sulfate proteoglycan 4 (CSPG4) promoter, and a SRY-box transcription factor 10 (SOX10) promoter, sequences of which are identified in Table 1 supra.

In any embodiment, cells of the population are transduced with a recombinant genetic construct encoding one or more neuronal reprogramming factors for producing neurons from glial progenitor cells selected from the group consisting of miR-9/9*, miR-124, an inhibitor of polypyrimidine-tract-binding protein 1 (PTBP1), an inhibitor of RE1-silencing transcription factor (REST), and/or one or more transcription factors selected from the group consisting of CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, SOX2, NEUROD1, NEUROD2, ISL1, LHX3, FOXG1, DLX5, NKX2.2, FEV, GATA2, LMX1B, FOXA2, and NGN2.

In accordance with this aspect of the disclosure, the recombinant genetic construct may be integrated into the chromosome of the one or more cells in the population. The term “integrated,” when used in the context of the recombinant genetic construct of the present disclosure means that the recombinant genetic construct is inserted into the genome or the genomic sequence of the one or more cells in the population. When integrated, the integrated recombinant genetic construct is replicated and passed along to daughter cells of a dividing cell in the same manner as the original genome of the cell.

### Methods of Generating Populations of Medium Spiny Neurons and Cortical Interneurons

Another aspect of the present disclosure relates to a method of generating a population of medium spiny neurons. This method involves providing a population of human glial progenitor cells; and expressing one or more medium spiny neuron reprogramming factors in the provided glial progenitor cell population under conditions suitable for medium spiny neuron production from the glial progenitor cells of the population.

In some embodiments of the methods of generating a population of medium spiny neurons disclosed herein, the human glial progenitor cells of the population are CD140⁺, CD44⁺, or CD140⁺/CD44⁺ cells.

In some embodiments, said expressing is carried out by administering a genetic construct comprising: a nucleic acid molecule encoding miR-9/9*, miR-124, or a combination thereof and an inducible promoter and/or operator system sequence operably linked to the nucleic acid molecule.

In some embodiments, said expressing is carried out by administering one or more genetic constructs comprising: a nucleic acid molecule encoding miR 124/9, ASCL1, and BRN2.

In some embodiments, said expressing further involves administering one or more genetic constructs comprising: a nucleic acid molecule encoding one or more medium spiny neuron transcription factors selected from the group consisting of CTIP2, DLX1, DLX2, and MYT1L, and an inducible promoter and/or operator system sequence operably linked to the nucleic acid molecule.

In some embodiments, the genetic construct is an expression vector. Suitable expression vectors are described in detail supra. Thus, in some embodiments, the expression vector is a viral vector. The viral vector may be selected from the group consisting of a lentiviral vector, an adeno-associated viral vector, vaccinia vector, and a retroviral vector.

Another aspect of the present disclosure relates to a method of generating a population of cortical interneurons. This method involves providing a population of human glial progenitor cells; and expressing one or more cortical interneuron reprogramming factors in the provided population of glial progenitor cells under conditions suitable for cortical interneuron production from the glial progenitor cells.

As described herein, the term “cortical interneurons” refers to a highly heterogeneous group of neurons with very diverse morphologies, connectivity, biochemistry, and physiological properties (Lim et al., “Development and Functional Diversification of Cortical Interneurons,” Neuron 100:294-313 (2018), which is hereby incorporated by reference in its entirety). These features are acquired during development through the implementation of specific transcriptional programs that are either intrinsically encoded or driven by interactions with the local microenvironment. The most prominent morphological characteristics of cortical interneurons are the shape and orientation of dendrites and axons. Biochemical properties define important aspects of synaptic communication, including the co-release of neuropeptides along with GABA. Intrinsic electrophysiological properties are largely determined by the combination of ionic channels they express. Interneurons establish synapses on different subcellular compartments of their targets, which impact their role in neural circuits.

In some embodiments, the cortical interneurons are selected from the group consisting of parvalbumin (PV)” interneurons (e.g., chandelier cells, basket cells, and translaminar interneurons), somatostatin (SST)” interneurons (e.g., Martinotti cells and non-Martinotti cells), and serotonin receptor 5HT3aR″ interneurons (e.g., vasoactive intestinal peptide “interneurons) (Lim et al., “Development and Functional Diversification of Cortical Interneurons,” Neuron 100:294-313 (2018), which is hereby incorporated by reference in its entirety). In some embodiments, the interneurons are GABAergic interneurons.

In some embodiments, the cortical interneuron reprogramming factors are selected from the group consisting of ASCL1, LMX1A, and NURR1. In accordance with such embodiments, said expressing is effective to produce fast-spiking parvalbumin (PV)-containing interneurons.

In some embodiments, the reprogramming factors are BRN2, ASCL1, MiR124/9, and LMX1A.

In some embodiments, the reprogramming factors are BRN2, ASCL1, miR124/9, and LHX6.

In some embodiments, the cortical interneuron reprogramming factors are selected from the group consisting of FOXG1, SOX2, ASCL1, DLX5, and LHX6. In accordance with such embodiments, said expressing is effective to produce GABAergic interneurons (see, e.g., Colasante et al., “Rapid Conversion of Fibroblasts into Functional Forebrain GABAergic Interneurons by Direct Genetic Reprogramming,” Stem Cell Stem 17:719-734 (2015), which is hereby incorporated by reference in its entirety).

In some embodiments, the cortical interneuron reprogramming factors are selected from the group consisting of NKX2.2, FEV, GATA2, LMX1B, ASCL1, and NGN2. In some embodiments, the cortical interneuron reprogramming factors are selected from the group consisting of ASCL1, FOXA2, LMX1B, and FEV. In accordance with such embodiments, said expressing is effective to produce seratonergic neurons (see, e.g., Vadodaria et al., “Generation of Functional Human Serotonergic Neurons from Fibroblasts,” Nature 21:49-61 (2016) and Xu et al., “Direct Conversion of Human Fibroblasts to Induced Serotonergic Neurons,” Mol. Psychiatry 21(1): 62-70 (2016), which are hereby incorporated by reference in their entirety).

In some embodiments of the methods of generating a population of medium spiny neurons disclosed herein, the human glial progenitor cells of the population are CD140⁺, CD44⁺, or CD140⁺/CD44⁺ cells.

### EXAMPLES

### Materials and Methods for Examples 1-3

Mice. 16 weeks old PDGFRa-CreER mice were used in Examples 1-3.

Viruses. Viruses were generated following standard molecular biology cloning, sequence verified. Viral particles were generated by co-transfection with lentivirus helper plasmid (pSPAX-2) viral envelope expressing plasmid (pLP-VSV-G) in HEK293FT cells. The viral preparations were concentrated by ultracentrifugation and were titered using a QPCR titration kit (Applied Biological Materials Inc; LV900).

Surgery. A viral cocktail was injected into the striatum at the following stereotaxic coordinates: Antero-posterior: +0.8 mm; Medio-latera: +/−1.8 mm and Dorso-ventral: −2.5 mm from dura). Tamoxifen was started on the date of injection, and was administered for 2 weeks (125 mg/kg weight). Animals were sacrificed 3 weeks post-injection.

Immunofluorescence staining. Immunofluorescence staining was carried using the following antibodies: anti NeuN (Sigma; ABN178); anti-Parvalbumin (Abcam; AB181086); DARPP32 (Abcam; AB40801); and EGFP (Bioss Inc; BS2194R).

### Example 1—Silencing Mouse PTBP Converts Glial Progenitor Cells into Neurons in Adult Mice

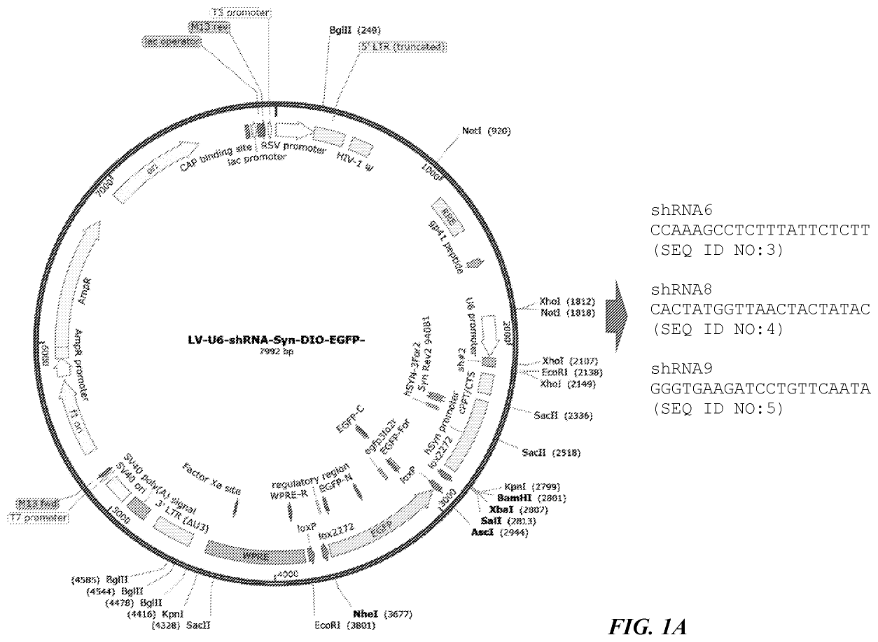

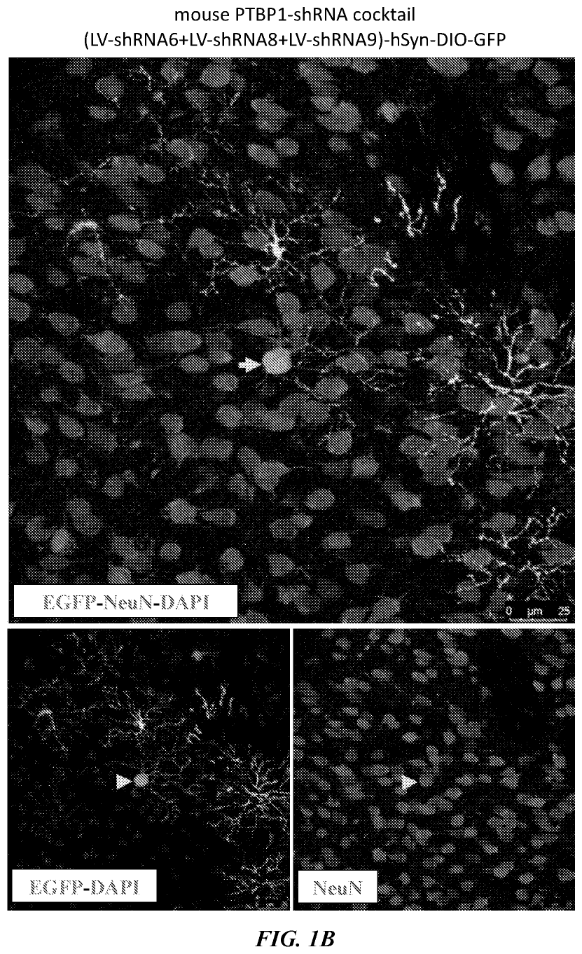

To investigate whether silencing mouse PTBP could convert glial progenitor cells into neurons in adult mice, lentiviral vectors expressing shRNA against mouse Ptbp1 gene and EGFP in a Cre-dependent matter under the neuron-specific synapsin promoter were designed (FIG. 1A). 16-week-old PDGFRa-CreER mice treated with a mixture of LV-shRNA6-Syn-DIO-EGFP, LV-shRNA8+LV-Syn-DIO-EGFP, and LV-shRNA9-Syn-DIO-EGFP showed that new neurons could be identified three weeks after viral injection (FIG. 1B). New neurons expressed the mature neuronal marker NeuN together with the PDGFRA-CreER-dependent EGFP (FIG. 1B), indicating that the NeuN” neurons arose from PDGFRA-defined glial progenitor cells. This strategy for fate mapping established that the BeuN “/EGFP” co-expressing neurons could only have been derived from the PDGFRA Cre-expressing glial progenitor cells.

### Example 2—Glial Progenitor Cells May be Converted into Medium Spiny Neurons by ASCL1 and BRN2 Overexpression with miR124/9

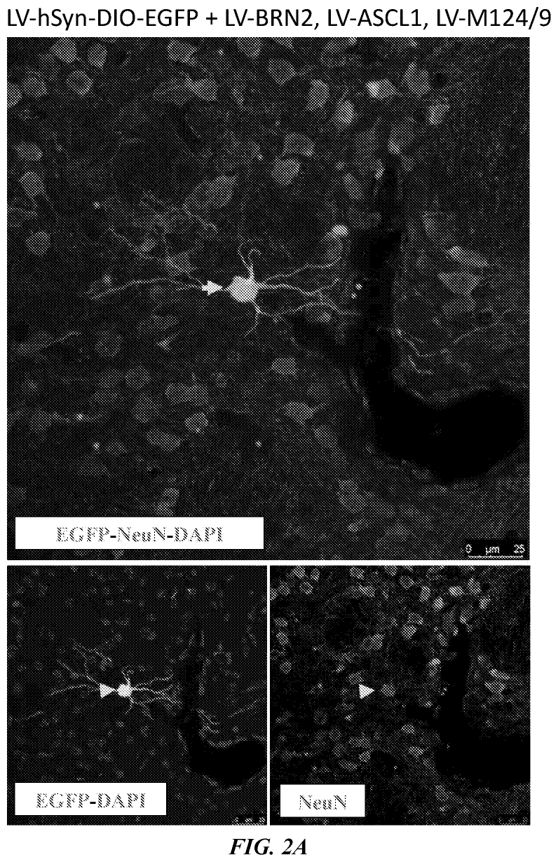

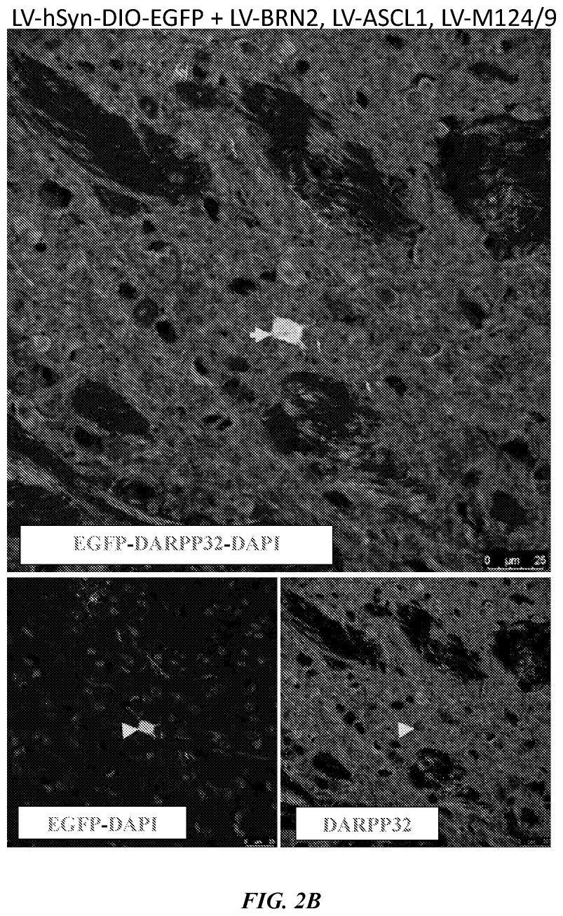

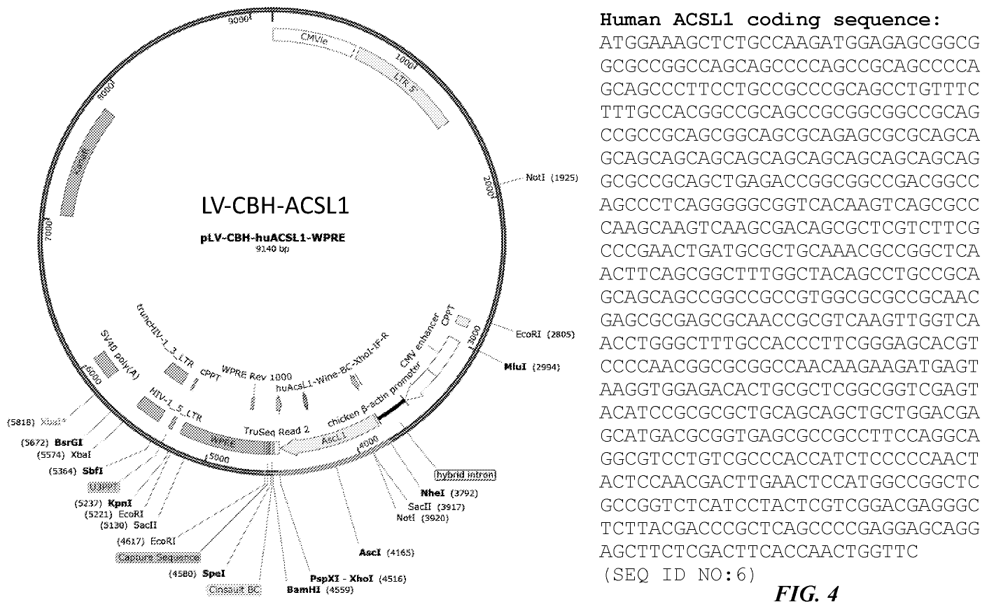

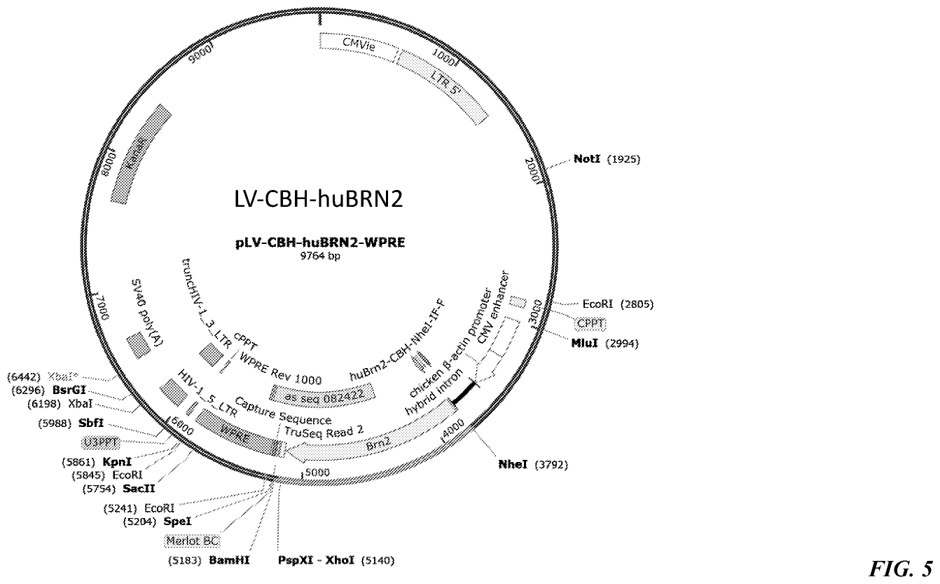

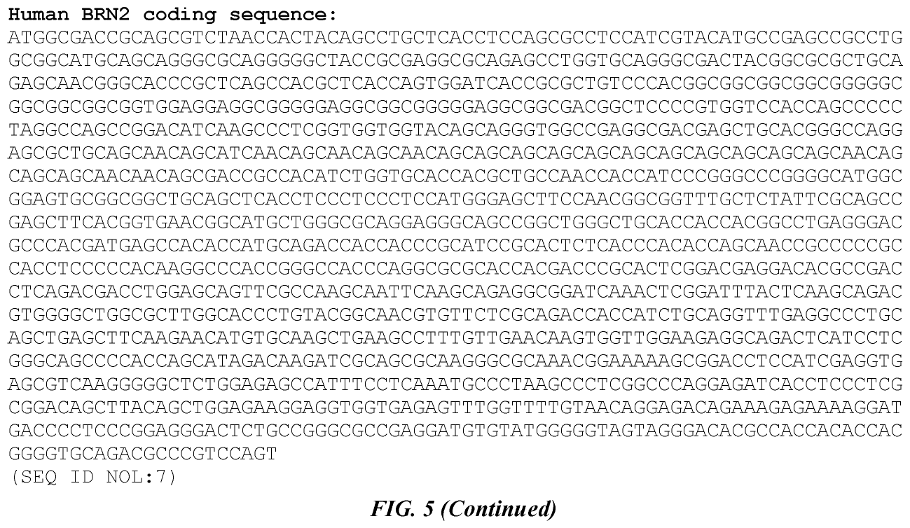

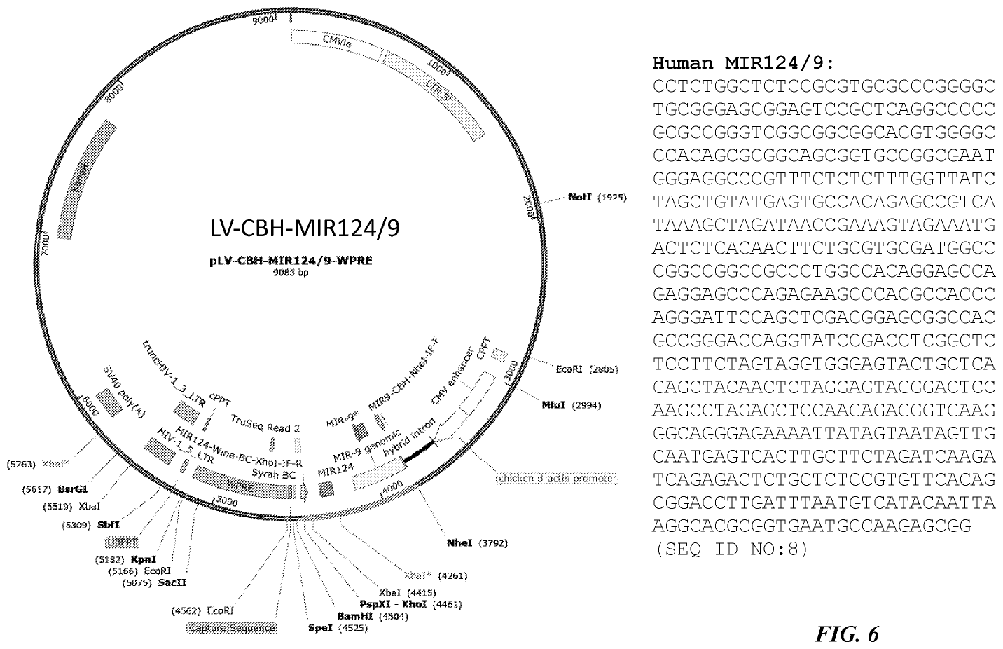

To investigate whether glial progenitor cells may be converted into medium spiny neurons, lentiviral vectors expressing ASCL1 (FIG. 4), huBRN2 (FIG. 5), and MIR 124/9 (FIG. 6) were designed. Forced expression of Ascl, BRN2, and MIR 124/9 using lentiviral vectors, combined with fate mapping using PDGFRA-CreER-dependent neuron-specific reporter expression demonstrated that new neurons could be generated from glial progenitor cells in the mouse brain. Briefly, a lentiviral mixture consisting of LV-huBRN2, LV-ASCL1, LV-M124/9, and LV-hSyn-DIO-EGFP was injected into the striatum of 16 week old PDGFRa-CreER mice. Mice were then treated with Tamoxifen for two weeks, to activate Cre translocation to the nucleus and recombinase-triggered EGFP expression. Mice were sacrificed a week later, three weeks post viral injection. Histological processing of the brain revealed new neurons expressing the mature neuronal marker NeuN together with EGFP, indicating the glial progenitor cell origin of these neurons, since in this system, Cre-dependent expression of EGFP can only occur in glial progenitor cells (FIG. 2A). A cohort of these newly generated, glial-derived neurons expressed DARPP2, indicative of their acquisition of a medium spiny neuronal phenotype (FIG. 2B).

### Example 3—Glial Progenitor Cells are Converted into Parvalbumin Interneurons in the Striatum

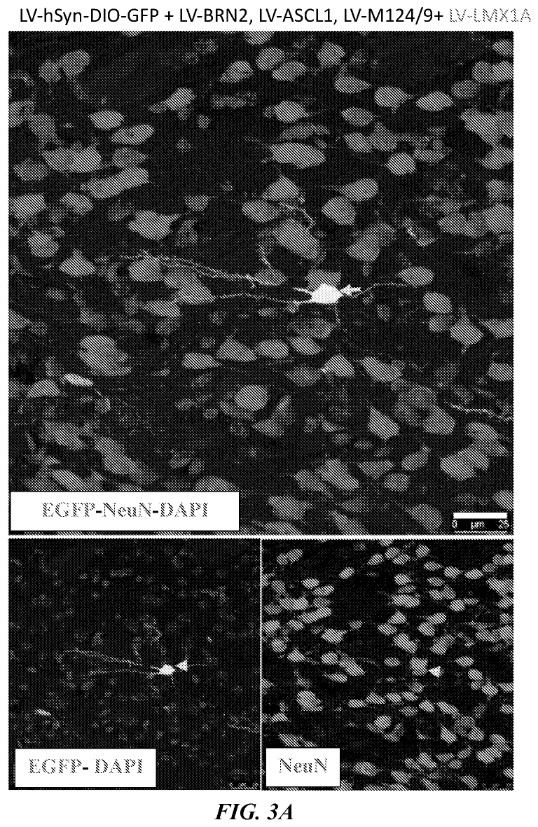

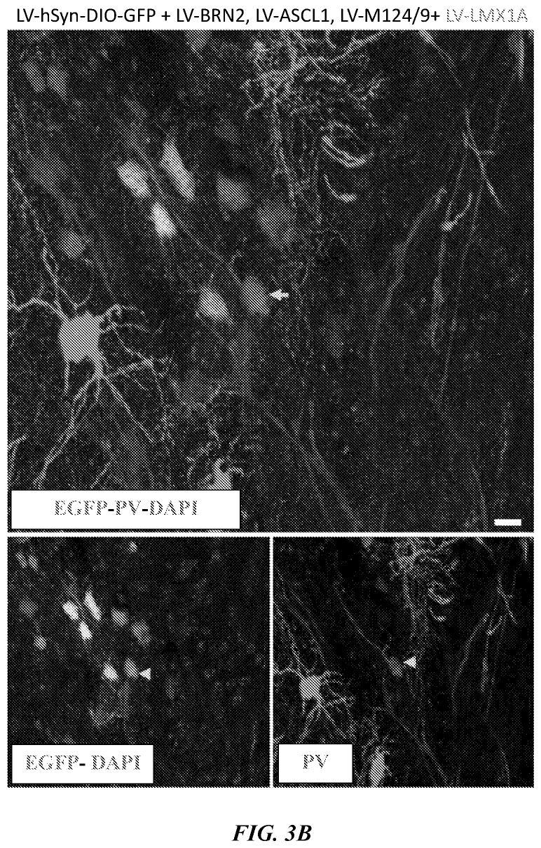

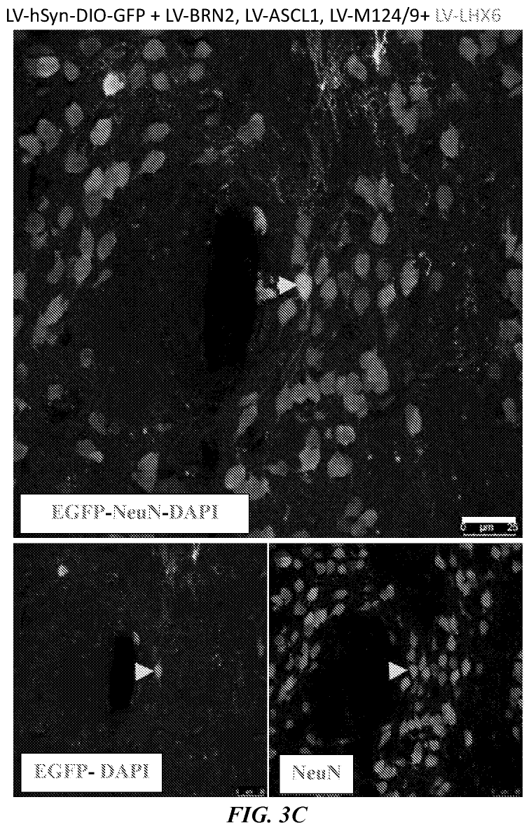

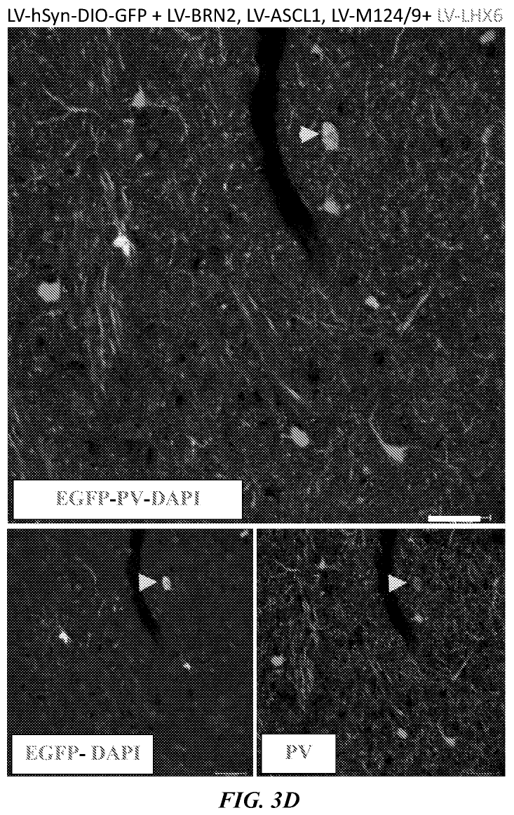

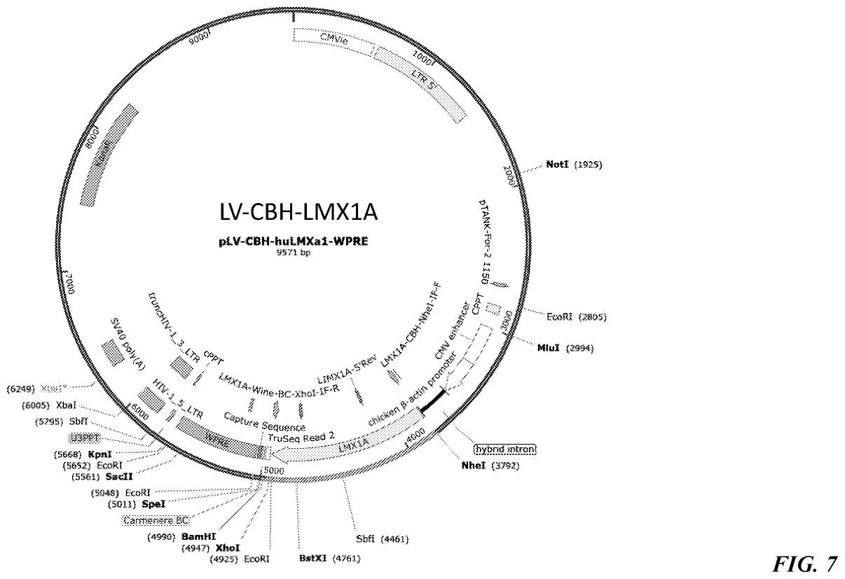

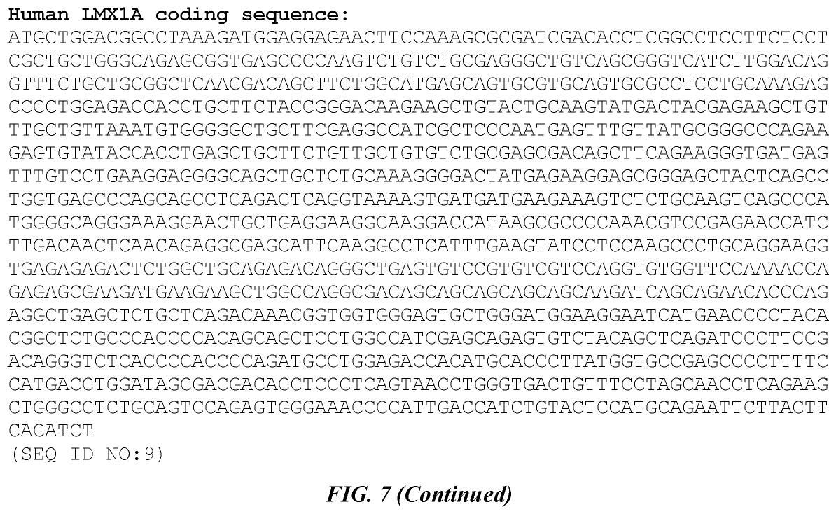

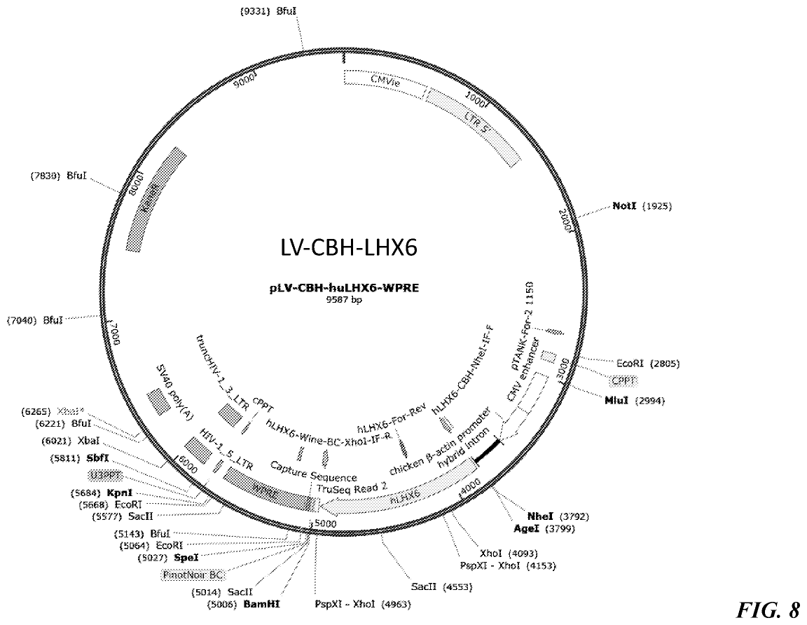

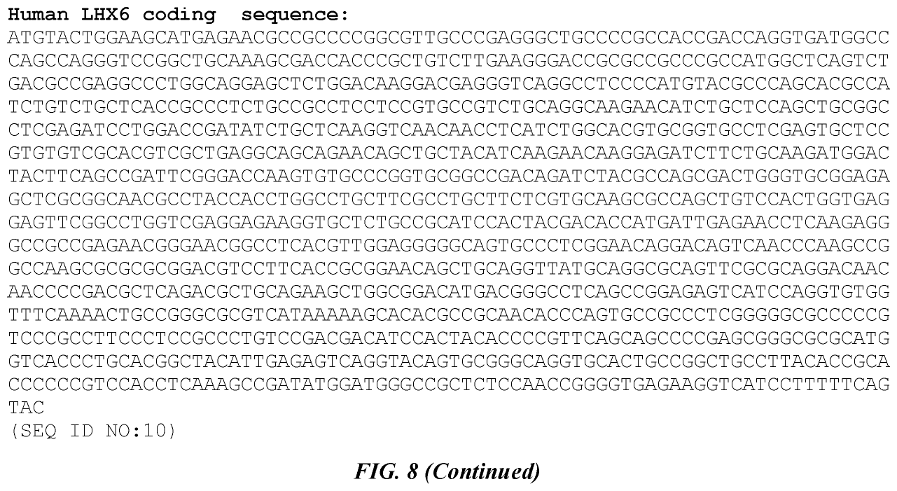

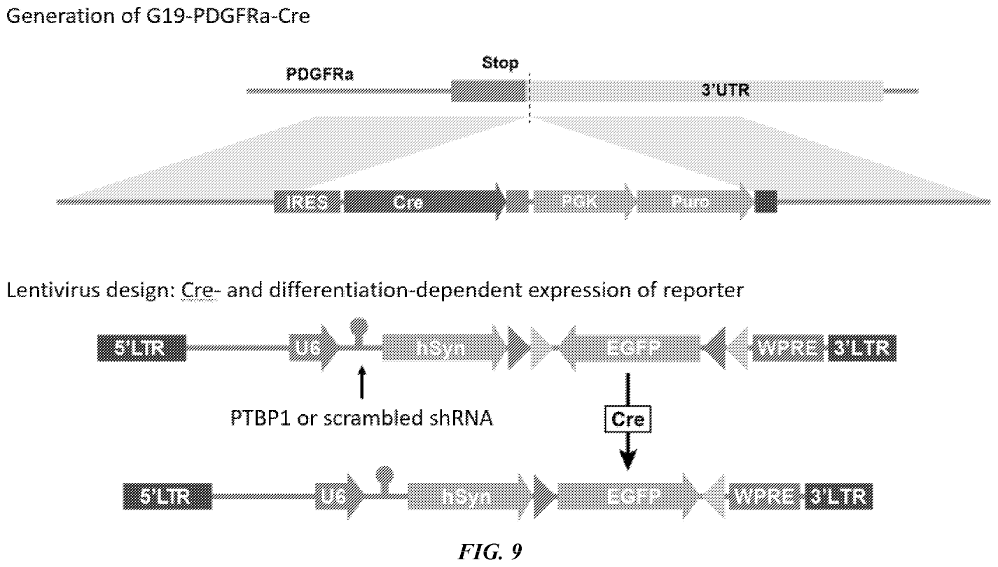

Next, whether glial progenitor cells may be converted into parvalbumin interneurons was investigated by combining direct differentiation of GPCs to neuronal phenotype with transcription factors involved in the differentiation of parvalbuminergic interneurons in development (i.e., LMX1A and/or LHX6). Briefly, a lentiviral mixture consisting of LV-huBRN2, LV-ASCL1, LV-M124/9, and LV-hSyn-DIO-EGFP, along with either LMX1A (FIGS. 3A-3B; FIG. 7) or LHX6 (FIGS. 3C-3D; FIG. 8) was injected into the striatum of 16-week-old PDGFRa-CreER mice. Following injection, mice were treated with Tamoxifen for two weeks, to activate Cre translocation to the nucleus and recombinase-triggered GFP expression. Mice were sacrificed a week later, three weeks post viral injection. Upon processing the brains of virally-treated PDGFRa-CreER mice, new NeuN-defined neurons were readily identified by of their expression of EGFP (FIG. 3A, FIG. 3C). A cohort of glial derived neurons also expressed Parvalbumin (PV) (FIG. 3B, FIG. 3D).

### Example 4—In Vitro Human Glial Progenitor Cells Phenoconversion to Neurons

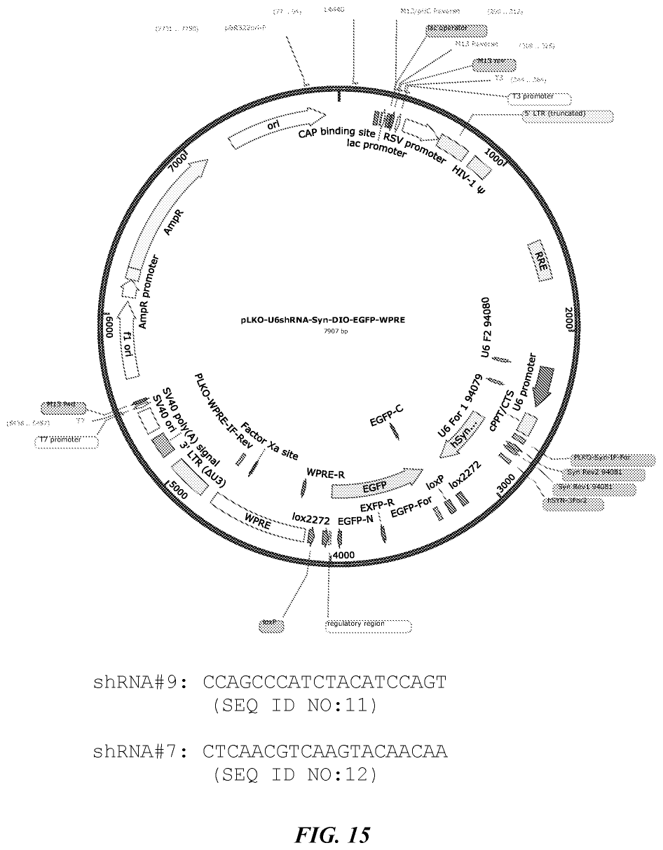

Whether human glial progenitor cells could be converted to neurons was investigated by designing lentiviral vectors for Cre- and differentiation-dependent expression of a reporter (EGFP) (FIG. 1; FIG. 15). Lentiviral vectors included PTBP1 shRNA under control of the U6 promoter (FIG. 1; FIG. 15). Viruses were generated following standard molecular biology cloning and sequence verified. Viral particles were generated by co-transfection with lentivirus helper plasmid (pSPAX-2) viral envelope expressing plasmid (pLP-VSV-G) in HEK293FT cells. The viral preparations were concentrated by ultracentrifugation and were titered using QPCR titration kit (Applied Biological Materials Inc; LV900).

Briefly, hESC Genea19-PDGFRa-Cre were induced into glial progenitor cells following a standard protocol. Cells were transduced with PTBP1 shRNA 7, PTBP1 shRNA 9, or control-expressing lentivirus at multiplicity of infection (MOI) of 1 in glial media. The following day, media was switched neuronal media M21 supplemented with BDNF 20 ng/ml. Media was changed every 48 hours. All cells were fixed with 4% paraformaldehyde seven days post viral transduction.

Immunofluorescence staining was carried using following antibodies: anti-MAP2 (Millipore; MAB3418); anti-NeuN (Sigma; ABN178); anti-EGFP (Bioss Inc; BS2194R) anti-HuC/HuD (Invitrogen; A-21271).

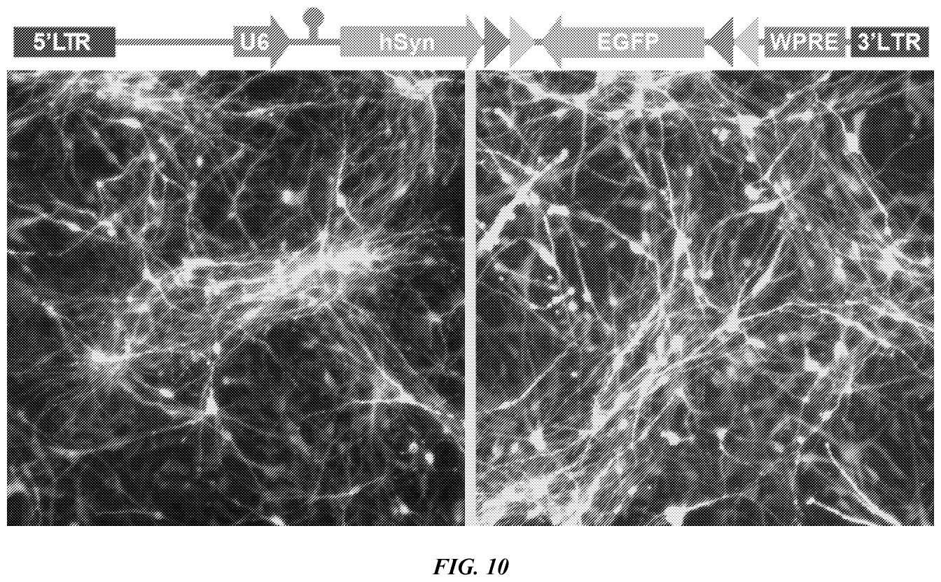

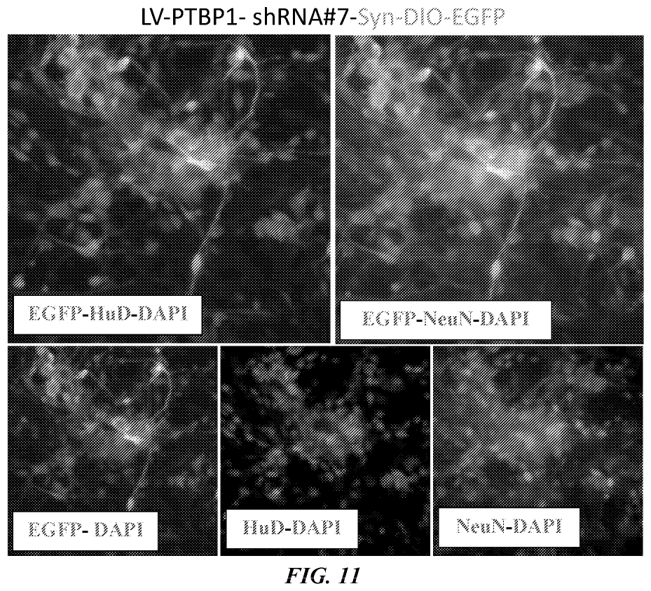

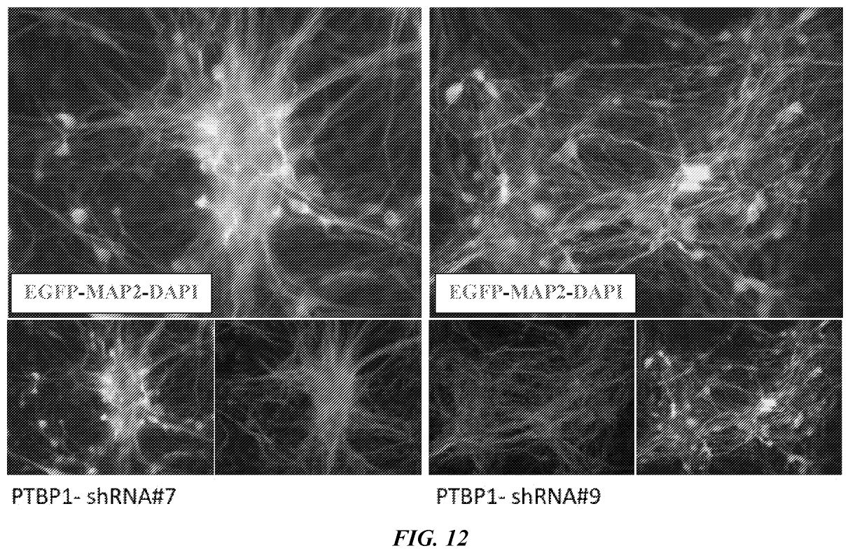

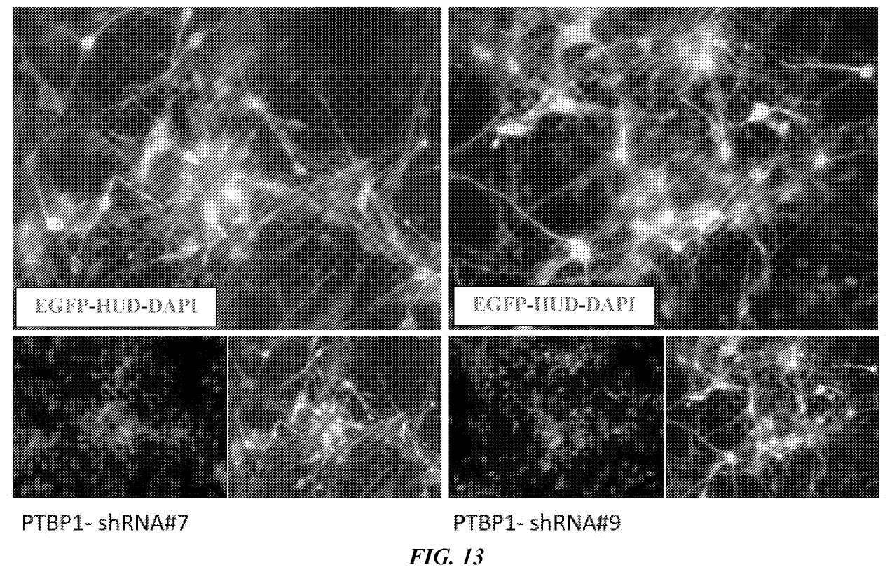

Genetic lineage tagging demonstrated that EGFP expression was restricted to neurons converted from human GPCs (FIG. 10). Synapsin promoter restricted expression of EGFP reporter was observed in HuD “/NeuN” neurons produced from PDGFRA-Cre expressing human glial progenitor cells (FIG. 11). Phenoconverted neurons expressed the mature neuronal markers MAP2 (FIG. 12) and HuD (FIG. 13).

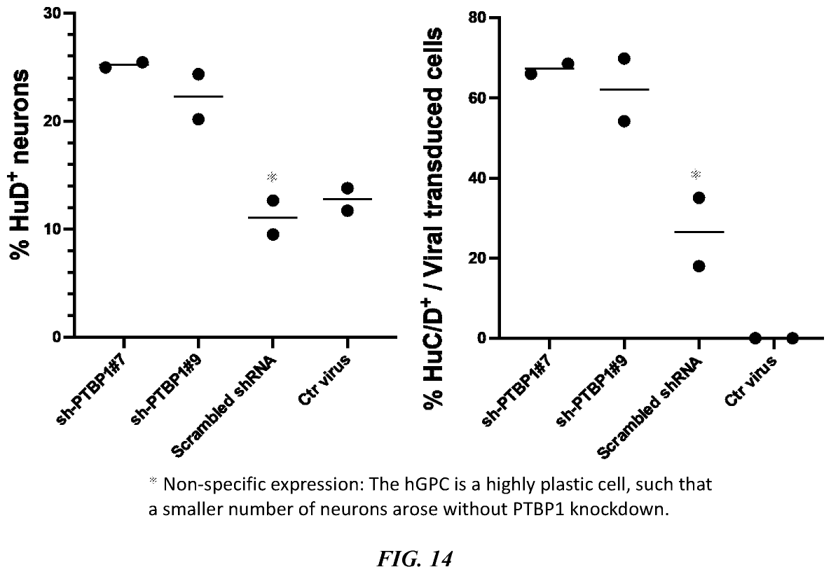

FIG. 14 demonstrates that PTBP1 knockdown using lentiviral vectors expressing shRNA 7 or shRNA 9 selectively converts human GPCs into neurons.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of generating a population of neurons, said method comprising:
providing a population of human glial progenitor cells;
providing a recombinant genetic construct comprising;
(i) a promoter and/or enhancer for a gene which is selectively or specifically expressed by human glial progenitor cells, and
(ii) a nucleic acid sequence encoding one or more neuronal reprogramming factors for producing neurons from glial progenitor cells, wherein said nucleic acid sequence is operably linked to the 3′ end of said promoter and/or enhancer to achieve expression of the one or more neuronal reprogramming factors;
transfecting cells of said glial progenitor cell population with the recombinant genetic construct; and
culturing the population after said transfecting under conditions suitable for neuron production from the transfected glial progenitor cells of the population.

2. The method of claim 1, wherein the glial progenitor cells of the population are CD140⁺, CD44⁺, or CD140⁺/CD44⁺ human glial progenitor cells.

3. A method of inducing the production of neurons in a subject in need thereof, said method comprising:
providing a recombinant genetic construct comprising:
(i) a promoter and/or enhancer for a gene which is selectively or specifically expressed by human glial progenitor cells, and
(ii) a nucleic acid sequence encoding one or more neuronal reprogramming factors for producing neurons from glial progenitor cells, wherein said nucleic acid sequence is operably linked to the 3′ end of said promoter and/or enhancer; and
administering, to the subject in need of neuron production, the recombinant genetic construct, under conditions effective for the one or more neuronal reprogramming factors to be expressed in glial progenitor cells of the subject, thereby inducing neuron generation in said subject.

4. The method of claim 3, wherein the subject has a neurodegenerative condition or disease.

5. A recombinant genetic construct comprising:
(i) a promoter and/or enhancer for a gene which is selectively or specifically expressed by human glial progenitor cells, and
(ii) a nucleic acid sequence encoding one or more neuronal reprogramming factors for producing neurons from glial progenitor cells, wherein said nucleic acid sequence is operably linked to the 3′ end of said promoter and/or enhancer to achieve expression of the one or more neuronal programming factors in glial progenitor cells.

6. The recombinant genetic construct of claim 5, wherein the gene selectively or specifically expressed by human glial progenitor cells is selected from the group consisting of PDGFRA, ZNF488, GPR17, OLIG2, CSPG4, and SOX10.

7. The recombinant genetic construct of claim 5, wherein the gene selectively or specifically expressed by glial progenitor cells is GPR17.

8. The recombinant genetic construct of claim 7, wherein the GPR17 promoter-inclusive regulatory element has the sequence of SEQ ID NO: 1 or SEQ ID NO: 2.

9. The recombinant genetic construct of claim 5, wherein the one or more neuronal reprogramming factors are selected from the group consisting of medium spiny neuron reprogramming factors, cortical interneuron reprogramming factors, dopaminergic neuron reprogramming factors, peripheral sensory neuron reprogramming factors, nonadrenergic neuronal reprogramming factors, cholinergic reprogramming factors, and spinal motor neuron reprogramming factors.

10. The recombinant genetic construct of claim 5, wherein the one or more neuronal reprogramming factors are selected from miR-9/9* and miR-124.

11. The recombinant genetic construct of claim 5, wherein the one or more neuronal reprogramming factors is an inhibitor of polypyrimidine-tract-binding protein 1 (PTBP1).

12. The recombinant genetic construct of claim 11, wherein the inhibitor of PTBP1 comprises a PTBP1 siRNA or PTBP1 shRNA, or comprises a PTBP1 guide RNA and Cas protein.

13. (canceled)

14. The recombinant genetic construct of claim 5, wherein the one or more neuronal reprogramming factors is an inhibitor of REI-silencing transcription factor (REST).

15. The recombinant genetic construct of claim 14, wherein the inhibitor of REST comprises a REST siRNA or REST shRNA, or comprises a REST guide RNA and Cas protein.

16. (canceled)

17. The recombinant genetic construct of claim 5, wherein the one or more neuronal reprogramming factors comprise one or more transcription factors selected from the group consisting of CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, SOX2, NEUROD1, NEUROD2, ISL1, LHX3, FOXGI, DLX5, NKX2.2, FEV, GATA2, LMX1B, FOXA2, and NGN2.

18-27. (canceled)

28. A population of human glial progenitor cells comprising the genetic construct of claim 5.

29. A method of generating a population of medium spiny neurons, said method comprising:
providing a population of human glial progenitor cells; and
expressing one or more medium spiny neuron reprogramming factors in the provided glial progenitor cell population under conditions suitable for medium spiny neuron production from the glial progenitor cells of the population.

30. (canceled)

31. The method of claim 29, wherein said expressing is carried out by administering a genetic construct comprising:
a nucleic acid molecule encoding miR-9/9*, miR-124, or a combination thereof and an inducible promoter and/or operator system sequence operably linked to the nucleic acid molecule.

32. The method of claim 31, wherein said expressing further comprises:
administering one or more genetic constructs comprising:
a nucleic acid molecule encoding one or more medium spiny neuron transcription factors selected from the group consisting of CTIP2, DLX1, DLX2, and MYT1L, and
an inducible promoter and/or operator system sequence operably linked to the nucleic acid molecule.

33-35. (canceled)

36. A method of generating a population of cortical interneurons, said method comprising:
providing a population of human glial progenitor cells; and
expressing one or more cortical interneuron reprogramming factors in the provided population of glial progenitor cells under conditions suitable for cortical interneuron production from the glial progenitor cells.

37. (canceled)


SEQUENCE LISTING (12 sequences)

SEQ ID NO: 1 - DNA, 2199 bp, synthetic construct
  source 1..2199: mol_type = other DNA
  regulatory 1..2199: regulatory_class = promoter
  misc_feature 1..2199: note = GPR17 Promoter
ggatacggaagagatccaatcgcagacccaagatccccacccaggttatggtgggcagaccccagatgccagggccacccattcagcatccctccctggaccccaggacctgctactgctgggtgtctggactccatcctgcacagcactgtgctccatctgccctggggtgtctcatcatcagctgtgtgcagggcaaggggcccaaacaaaggcccagcagtcactggctaagctgccgactggctctctgtgcctccccaagaccctatgtgcccagcagggggcaacagctcagggtcagctgaccgaatgcctcggtgaatgaatgactctacaagagaggaagggagcctcggtgggcatcatctcccctcgactactggccagagccctggctcttacaccccagcgacgggaagcagttgtggcctgtggcttcagtcttcatcaccacaatccctgaagcccacccttgcccagacacctgtgccccagccccaaccccaggccacctcctcagcaggtctggggctgagctgccccacctggcgcctatggcggccagcccatgccccctgcggtgcctctgtcccagactcagcatgtaggccccatgaccccactccacattctggtgactcctcctgagcgtcaggacaacactcaacccacgaggaattatttctgtctcaaagatgcaggaatcagctcaacgcctcaaaactccatcaccacggtcaatgcccttgaagccatcgacagtgatcaccccaataacagaaggtctgtgagcccagaaatgccctgctcagggtggttagcttcaagccaccacctttccaaccagcctgggccagttcttccagacagccgcctgcgggcacaacaggaaagagacctgcgccccggctcagacacctcacacccagctggctctcaggccagacaaactgggaagcccatctctcttgaaggaagtccagatgggaaacagcttctcaacagaccagatcacagcatcagatctaaaggtggccttcagaattctttttcaggttgaattaggatcaaatctaagaattctaaattcaaaatgcagcagaaaaacaaaacacacacacacacggagcctaagttctggagtgacatgtgcttgggttcaaatcctggctctgttgcttcctactgtttgttgatgggtgagtttcttcatttgcctgagcctcagtttccttgtctgtaaaatggggcaataatcccagctgcacagggtgatgtgaagagacaaatttaagacactgccccttaaatgctagccacatacatacagttttcaatgtttaaacaacaaaatgtaaagtcttttgaaaccaggaagggtgatttggtttcccatgttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaagggc

SEQ ID NO: 2 - DNA, 799 bp, synthetic construct
  source 1..799: mol_type = other DNA
  regulatory 1..799: regulatory_class = promoter
  misc_feature 1..799: note = GPR17 Promoter
gttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaaggg

SEQ ID NO: 3 - RNA, 21 nt, synthetic construct
  source 1..21: mol_type = other RNA
  misc_feature 1..21: note = shRNA 6
ccaaagcctctttattctctt

SEQ ID NO: 4 - RNA, 21 nt, synthetic construct
  source 1..21: mol_type = other RNA
  misc_feature 1..21: note = shRNA 8
cactatggttaactactatac

SEQ ID NO: 5 - RNA, 21 nt, synthetic construct
  source 1..21: mol_type = other RNA
  misc_feature 1..21: note = shRNA 9
gggtgaagatcctgttcaata

SEQ ID NO: 6 - DNA, 708 bp, synthetic construct
  source 1..708: mol_type = other DNA
  misc_feature 1..708: note = ASCL1
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttc

SEQ ID NO: 7 - DNA, 1336 bp, synthetic construct
  source 1..1336: mol_type = other DNA
  misc_feature 1..1336: note = BRN2
atggcgaccgcagcgtctaaccactacagcctgctcacctccagcgcctccatcgtacatgccgagccgcctggcggcatgcagcagggcgcagggggctaccgcgaggcgcagagcctggtgcagggcgactacggcgcgctgcagagcaacgggcacccgctcagccacgctcaccagtggatcaccgcgctgtcccacggcggcggcggcgggggcggcggcggcggtggaggaggcgggggaggcggcgggggaggcggcgacggctccccgtggtccaccagccccctaggccagccggacatcaagccctcggtggtggtacagcagggtggccgaggcgacgagctgcacgggccaggagcgctgcagcaacagcatcaacagcaacagcaacagcagcagcagcagcagcagcagcagcagcagcaacagcagcagcaacaacagcgaccgccacatctggtgcaccacgctgccaaccaccatcccgggcccggggcatggcggagtgcggcggctgcagctcacctccctccctccatgggagcttccaacggcggtttgctctattcgcagccgagcttcacggtgaacggcatgctgggcgcaggagggcagccggctgggctgcaccaccacggcctgagggacgcccacgatgagccacaccatgcagaccaccacccgcatccgcactctcacccacaccagcaaccgcccccgccacctcccccacaaggcccaccgggccacccaggcgcgcaccacgacccgcactcggacgaggacacgccgacctcagacgacctggagcagttcgccaagcaattcaagcagaggcggatcaaactcggatttactcaagcagacgtggggctggcgcttggcaccctgtacggcaacgtgttctcgcagaccaccatctgcaggtttgaggccctgcagctgagcttcaagaacatgtgcaagctgaagcctttgttgaacaagtggttggaagaggcagactcatcctcgggcagccccaccagcatagacaagatcgcagcgcaagggcgcaaacggaaaaagcggacctccatcgaggtgagcgtcaagggggctctggagagccatttcctcaaatgccctaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtatgggggtagtagggacacgccaccacaccacggggtgcagacgcccgtccagt

SEQ ID NO: 8 - RNA, 558 nt, synthetic construct
  source 1..558: mol_type = other RNA
  misc_feature 1..558: note = MIR124/9
cctctggctctccgcgtgcgcccggggctgcgggagcggagtccgctcaggcccccgcgccgggtcggcggcggcacgtggggcccacagcgcggcagcggtgccggcgaatgggaggcccgtttctctctttggttatctagctgtatgagtgccacagagccgtcataaagctagataaccgaaagtagaaatgactctcacaacttctgcgtgcgatggcccggccggccgccctggccacaggagccagaggagcccagagaagcccacgccacccagggattccagctcgacggagcggccacgccgggaccaggtatccgacctcggctctccttctagtaggtgggagtactgctcagagctacaactctaggagtagggactccaagcctagagctccaagagagggtgaagggcagggagaaaattatagtaatagttgcaatgagtcacttgcttctagatcaagatcagagactctgctctccgtgttcacagcggaccttgatttaatgtcatacaattaaggcacgcggtgaatgccaagagcgg

SEQ ID NO: 9 - DNA, 1146 bp, synthetic construct
  source 1..1146: mol_type = other DNA
  misc_feature 1..1146: note = LMX1A
atgctggacggcctaaagatggaggagaacttccaaagcgcgatcgacacctcggcctccttctcctcgctgctgggcagagcggtgagccccaagtctgtctgcgagggctgtcagcgggtcatcttggacaggtttctgctgcggctcaacgacagcttctggcatgagcagtgcgtgcagtgcgcctcctgcaaagagcccctggagaccacctgcttctaccgggacaagaagctgtactgcaagtatgactacgagaagctgtttgctgttaaatgtgggggctgcttcgaggccatcgctcccaatgagtttgttatgcgggcccagaagagtgtataccacctgagctgcttctgttgctgtgtctgcgagcgacagcttcagaagggtgatgagtttgtcctgaaggaggggcagctgctctgcaaaggggactatgagaaggagcgggagctactcagcctggtgagcccagcagcctcagactcaggtaaaagtgatgatgaagaaagtctctgcaagtcagcccatggggcagggaaaggaactgctgaggaaggcaaggaccataagcgccccaaacgtccgagaaccatcttgacaactcaacagaggcgagcattcaaggcctcatttgaagtatcctccaagccctgcaggaaggtgagagagactctggctgcagagacagggctgagtgtccgtgtcgtccaggtgtggttccaaaaccagagagcgaagatgaagaagctggccaggcgacagcagcagcagcagcaagatcagcagaacacccagaggctgagctctgctcagacaaacggtggtgggagtgctgggatggaaggaatcatgaacccctacacggctctgcccaccccacagcagctcctggccatcgagcagagtgtctacagctcagatcccttccgacagggtctcaccccaccccagatgcctggagaccacatgcacccttatggtgccgagccccttttccatgacctggatagcgacgacacctccctcagtaacctgggtgactgtttcctagcaacctcagaagctgggcctctgcagtccagagtgggaaaccccattgaccatctgtactccatgcagaattcttacttcacatct

SEQ ID NO: 10 - DNA, 1155 bp, synthetic construct
  source 1..1155: mol_type = other DNA
  misc_feature 1..1155: note = LHX6
atgtactggaagcatgagaacgccgccccggcgttgcccgagggctgccccgccaccgaccaggtgatggcccagccagggtccggctgcaaagcgaccacccgctgtcttgaagggaccgcgccgcccgccatggctcagtctgacgccgaggccctggcaggagctctggacaaggacgagggtcaggcctccccatgtacgcccagcacgccatctgtctgctcaccgccctctgccgcctcctccgtgccgtctgcaggcaagaacatctgctccagctgcggcctcgagatcctggaccgatatctgctcaaggtcaacaacctcatctggcacgtgcggtgcctcgagtgctccgtgtgtcgcacgtcgctgaggcagcagaacagctgctacatcaagaacaaggagatcttctgcaagatggactacttcagccgattcgggaccaagtgtgcccggtgcggccgacagatctacgccagcgactgggtgcggagagctcgcggcaacgcctaccacctggcctgcttcgcctgcttctcgtgcaagcgccagctgtccactggtgaggagttcggcctggtcgaggagaaggtgctctgccgcatccactacgacaccatgattgagaacctcaagagggccgccgagaacgggaacggcctcacgttggagggggcagtgccctcggaacaggacagtcaacccaagccggccaagcgcgcgcggacgtccttcaccgcggaacagctgcaggttatgcaggcgcagttcgcgcaggacaacaaccccgacgctcagacgctgcagaagctggcggacatgacgggcctcagccggagagtcatccaggtgtggtttcaaaactgccgggcgcgtcataaaaagcacacgccgcaacacccagtgccgccctcgggggcgcccccgtcccgccttccctccgccctgtccgacgacatccactacaccccgttcagcagccccgagcgggcgcgcatggtcaccctgcacggctacattgagagtcaggtacagtgcgggcaggtgcactgccggctgccttacaccgcaccccccgtccacctcaaagccgatatggatgggccgctctccaaccggggtgagaaggtcatcctttttcagtac

SEQ ID NO: 11 - RNA, 20 nt, synthetic construct
  source 1..20: mol_type = other RNA
  misc_feature 1..20: note = shRNA 9
ccagcccatctacatccagt

SEQ ID NO: 12 - RNA, 20 nt, synthetic construct
  source 1..20: mol_type = other RNA
  misc_feature 1..20: note = shRNA 7
ctcaacgtcaagtacaacaa